(19) United States
(12) Patent Application Publication
(10) Pub. No.: US 2016/0317582 A1
(43) Pub. Date: Nov. 3, 2016

(54) Characterization Of Bone Marrow Aspirate Concentrate

(71) Applicants: John Garrett Kennedy, New York, NY (US); Lisa Ann Fortier, Ithaca, NY (US); Brian Roy Barnes, Southborough, MA (US)

(72) Inventors: John Garrett Kennedy, New York, NY (US); Lisa Ann Fortier, Ithaca, NY (US); Brian Roy Barnes, Southborough, MA (US)

(21) Appl. No.: 14/938,735

(22) Filed: Nov. 11, 2015

Related U.S. Application Data
(60) Provisional application No. 62/077,975, filed on Nov. 11, 2014.

(51) Int. Cl. A61K 35/28; A61K 35/16; A61K 9/00; A61K 35/19

(52) U.S. Cl. CPC A61K 35/28; A61K 35/19; A61K 35/16; A61K 9/0019

(57) ABSTRACT

The disclosure relates to bone marrow aspirate concentrate and the use of the same.

### CROSS-REFERENCE TO RELATED APPLICATIONS

This application claims priority to, and the benefit of, U.S. Provisional Application No. 62/077,975, filed on Nov. 11, 2014, which is incorporated herein by reference in its entirety

### FIELD

The present invention relates generally to bone marrow concentrate, and the use thereof.

### BACKGROUND

Osteoarthritis (OA) is a leading cause of disability with aging and affects over 27 million people in the United States. OA is characterized by progressive degeneration of the cartilage at the ends of the bones. However, there is no effective treatment for OA.

Interleukin receptor antagonist protein (IRAP, e.g., IL-1ra) is a naturally-occurring, anti-inflammatory protein produced by monocytes and neutrophils. It has been reported IRAP competitively binds to the receptor of interleukin 1β, thereby preventing IL-1β from mediating inflammation. Autologous conditioned serum (ACS) that includes IRAP and is an injectable antiarthritic derived from the patient's own blood (marketed under the trade name Orthokine®) has been shown to treat OA effectively in Europe. However, ACS preparation requires a long incubation and multi-step of manipulation.

The technical problem on which the present invention is based is thus to provide methods and means for quick and easy production of an autologous IRAP concentrate that comprises a high concentration of IRAP (e.g., IL-1Ra), which serve as safe, cost-effective alternatives which can be carried out quickly for the use.

The invention solves this problem by providing a quick and easy method for producing a bone marrow concentrate that includes a high concentration of IRAP, which can be used for prophylactic or therapeutic treatment of the human or animal body.

### SUMMARY

The invention described herein addresses the problem of soft tissue healing and the failure to date of any effective therapeutics that can be prepared quickly and easily.

Accordingly, provided herein is a method for preparing a bone marrow concentrate. The method includes (1) collecting a bone marrow sample in a subject; and (2) concentrating the bone marrow sample, thereby preparing a bone marrow concentrate, where the bone marrow concentrate includes an autologous interleukin receptor antagonist protein (IRAP) concentrate comprising at least about 10,000 pg/mL of IRAP.

In some embodiments, the concentrating step is performed via centrifugation.

In some embodiments, the centrifugation is performed with a sensor. For example, the sensor is a light sensor or a laser sensor that detects fluid density change.

In some embodiments, the IRAP has a concentration of at least about 12,000 pg/mL, about 15,000 pg/mL or more.

In some embodiments, the bone marrow concentrate further includes cytokine(s). For example, the cytokine is PDGF, TGF-β1, TGF-β2, TGF-β3, IL-1β, IL-8, or any combination thereof.

In some embodiments, the bone marrow sample is collected in the presence of at least one anticoagulant agent. For example, the anticoagulant agent comprises acid citrate dextrose solution (ACD) or Heparin.

Also provided herein is a method for facilitating soft tissue healing in a subject in need thereof by administering to the subject a therapeutically effective amount of a composition having the bone marrow concentrate prepared according to the method for preparing a bone marrow concentrate.

In some embodiments, the composition is administered locally.

In some embodiments, the composition is administered via local injection.

In some embodiments, the soft tissue is cartilage.

In some embodiments, the method further includes locally administering to the subject a therapeutically effective amount of a second composition having a platelet rich plasma (PRP).

Also provided herein is a method for treating osteoarthritis in a subject in need thereof by administering to the subject a therapeutically effective amount of a composition having the bone marrow concentrate prepared according to the method for preparing a bone marrow concentrate.

In some embodiments, the composition is administered locally.

Also provided herein is a method for facilitating skin regeneration in a subject in need thereof by administering to the subject a therapeutically effective amount of a composition having the bone marrow concentrate prepared according to the method for preparing a bone marrow concentrate.

In some embodiments, the composition is administered locally.

Also provided herein is a method for treating inflammation in a subject in need thereof by administering to the subject a therapeutically effective amount of a composition having the bone marrow concentrate prepared according to the method for preparing a bone marrow concentrate.

In some embodiments, the composition is administered locally.

Also provided herein is a composition having a bone marrow concentrate, where the bone marrow concentrate is prepared according to the method for preparing a bone marrow concentrate.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents and other references mentioned herein are incorporated by reference in their entirety for all purposes. The references cited herein are not admitted to be prior art to the claimed invention. In the case of conflict, the present specification, including definitions, will control. In addition, the materials, methods and examples are illustrative only and are not intended to be limiting.

Other features and advantages of the invention will be apparent from the following detailed description and claims.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations.

FIG. 1 is a representative bar graph showing platelet counts for whole blood and platelet rich plasma (PRP) from the Arteriocyte Magellan® System.

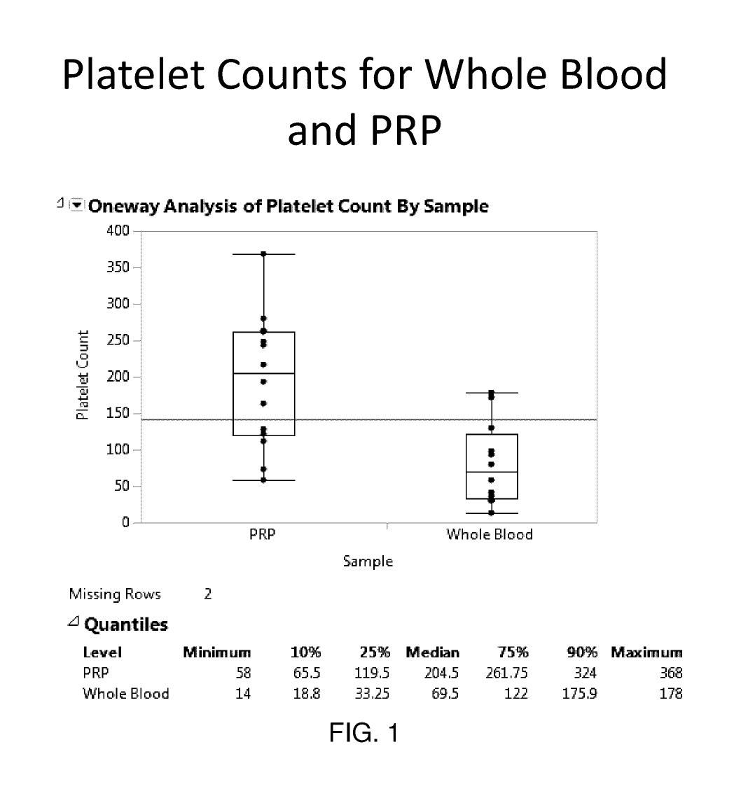

FIGS. 2A-2B are representative curve graphs showing platelet count distributions in (FIG. 2A) whole blood and (FIG. 2B) PRP.

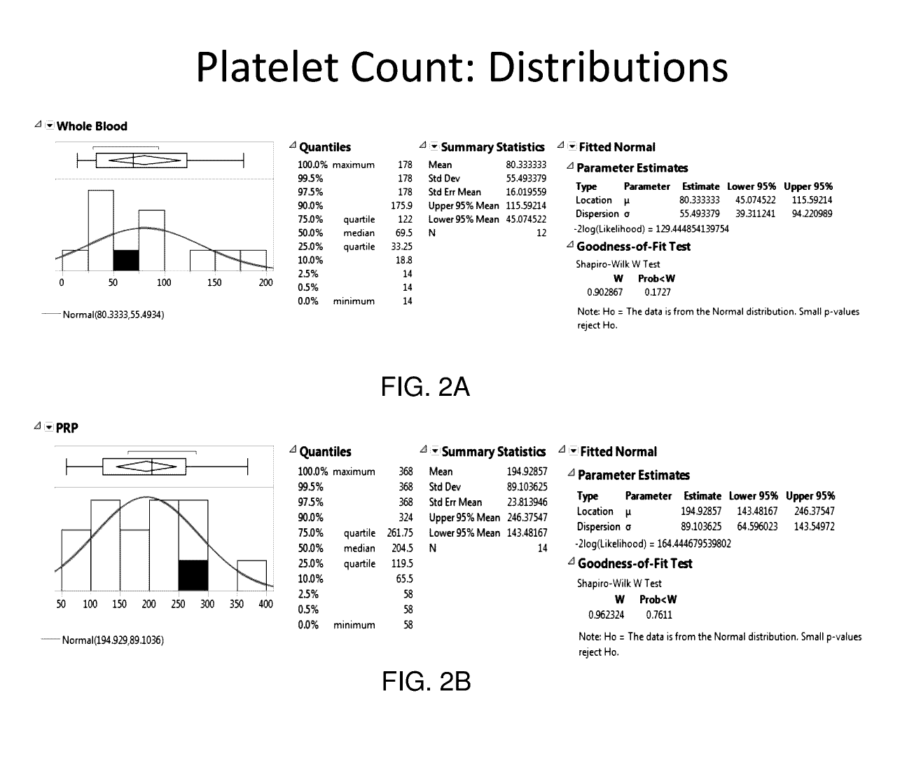

FIGS. 3A-3B are representative graphs showing platelet count in matched pairs and difference in distribution. FIG. 3A shows the difference between PRP and whole blood, and FIG. 3B shows difference in distribution.

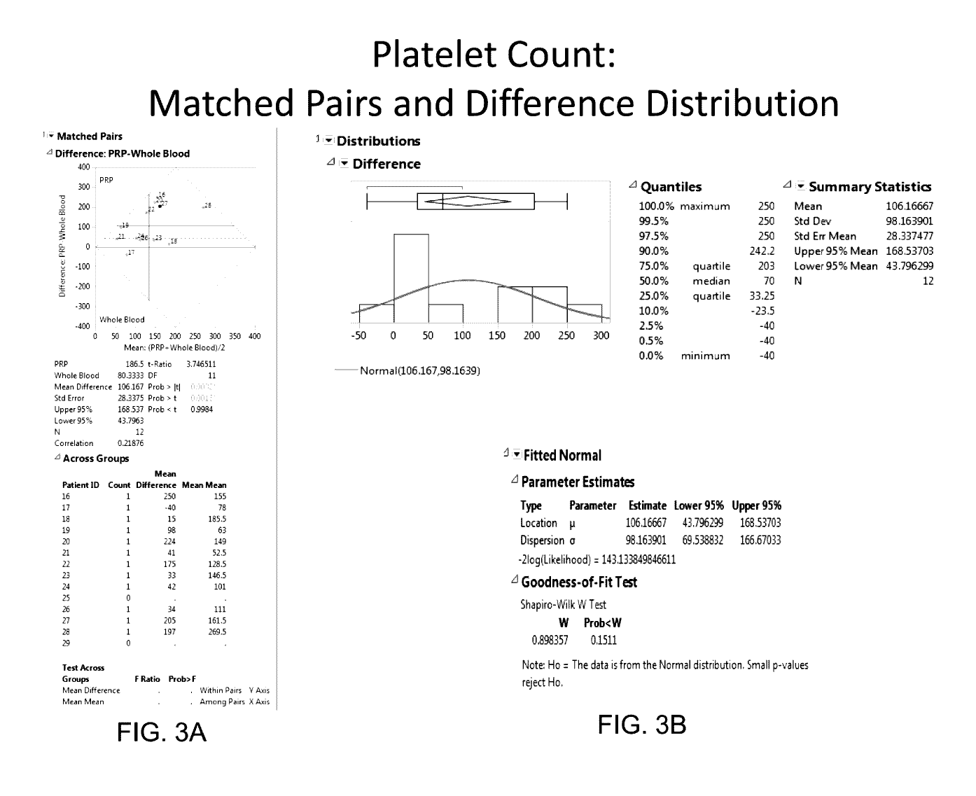

FIG. 4 is a representative bar graph showing platelet counts for whole blood and platelet rich plasma (PRP). Matched Pairs, paired T test: Prob>t meaning that the difference between PRP-Whole Blood Platelet Counts is statistically significant. Specifically that there is an increase from Whole Blood to PRP.

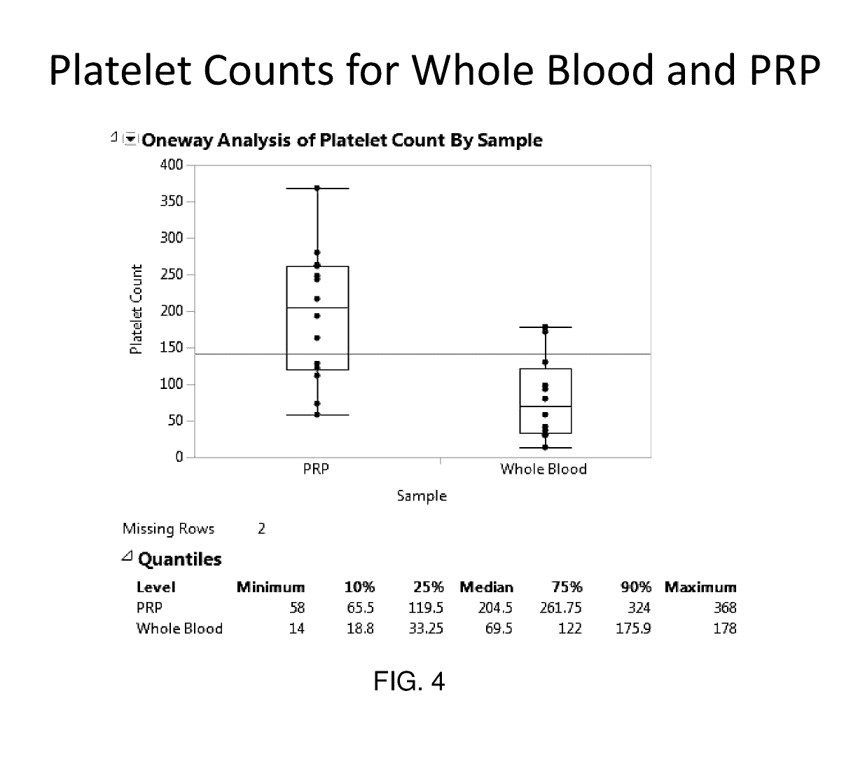

FIG. 5 is a representative bar graph showing platelet counts for bone marrow aspirate (BMA), and bone marrow concentrate (BMC) from two commercially available centrifuges; BMC-X (by SmartPrep® 2) and BMC-Y (by Arteriocyte Magellan® System).

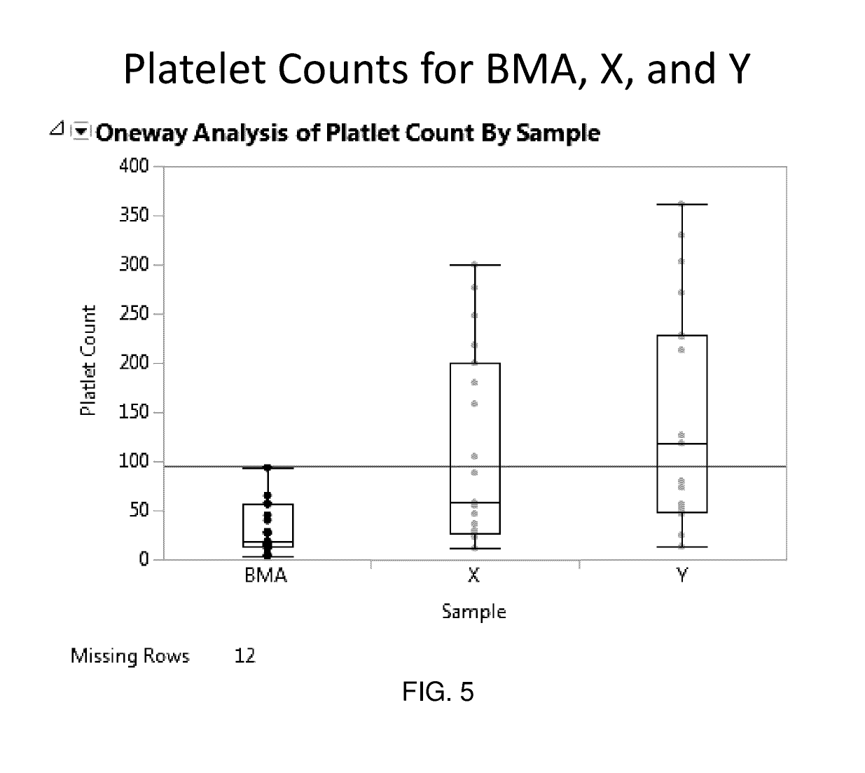

FIGS. 6A-6C are representative graphs showing platelet count distributions of (FIG. 6A) BMA, (FIG. 6B) BMC-X, and (FIG. 6C) BMC-Y.

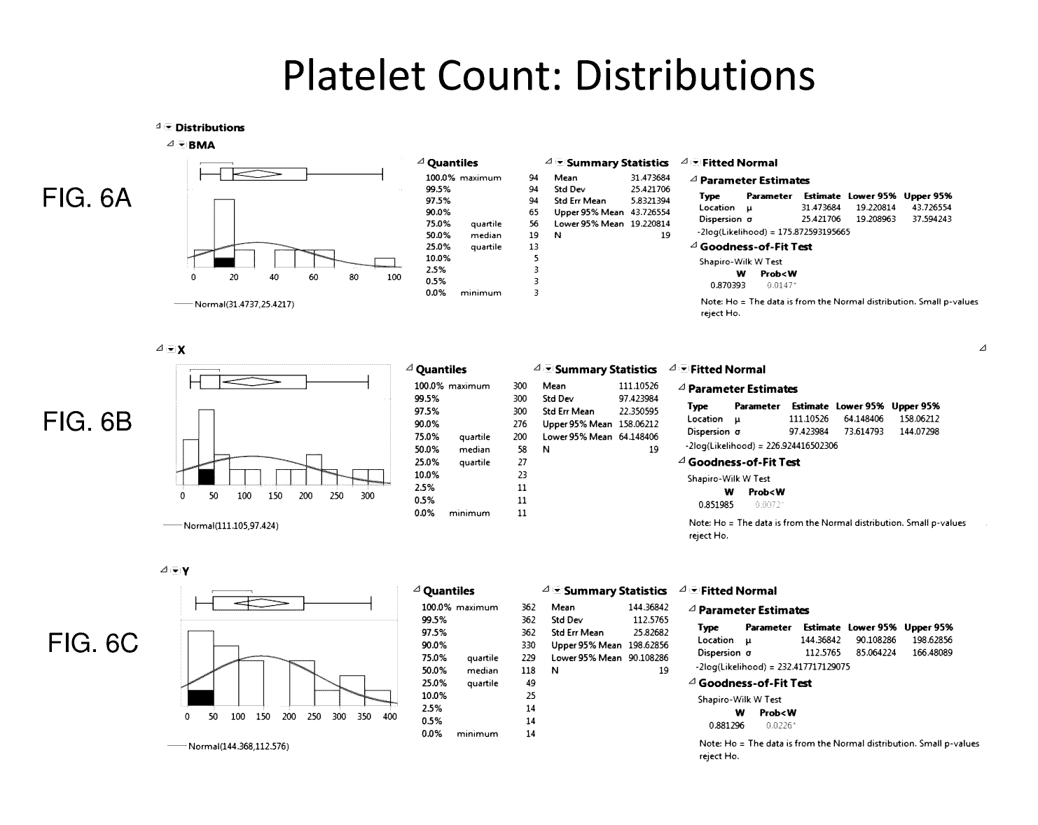

FIGS. 7A-7C are representative graphs showing difference in platelet count distributions of (FIG. 7A) BMC-X vs BMA; (FIG. 7B) BMC-Y vs BMA; and (FIG. 7C) BMC-X vs BMC-Y.

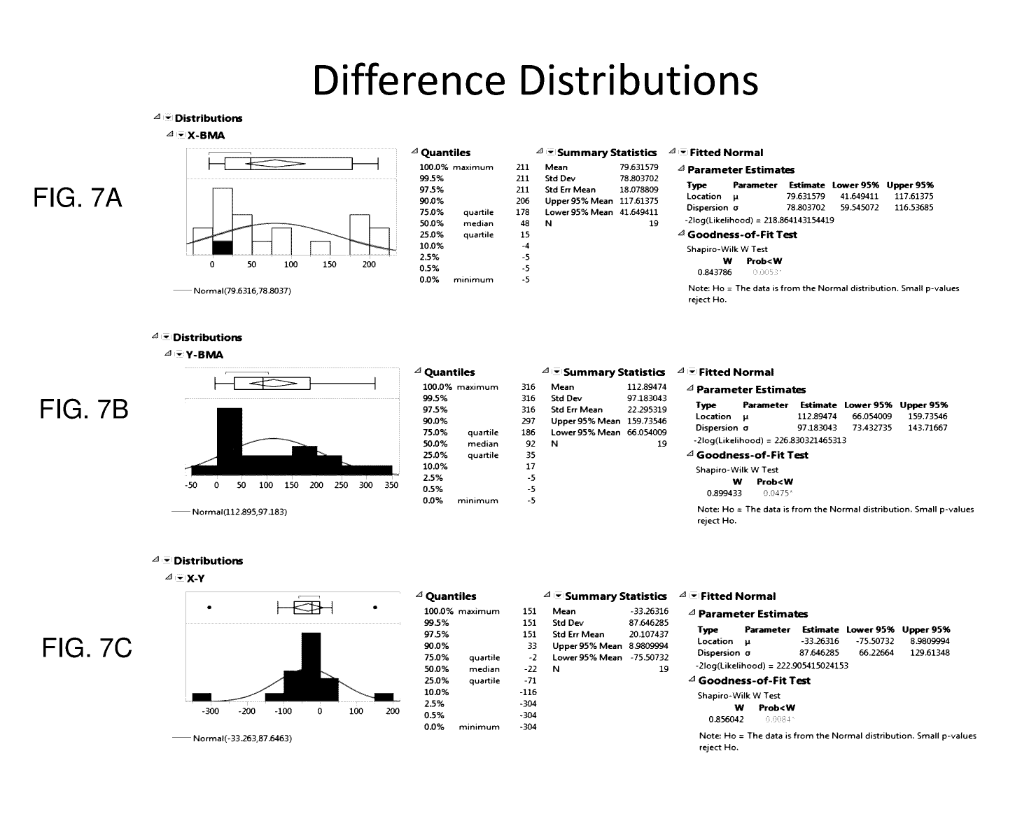

FIGS. 8A-8C are representative graphs showing difference in distributions of matched pairs in (FIG. 8A) BMC-X vs BMA; (FIG. 8B) BMC-Y vs BMA; and (FIG. 8C) BMC-X vs BMC-Y.

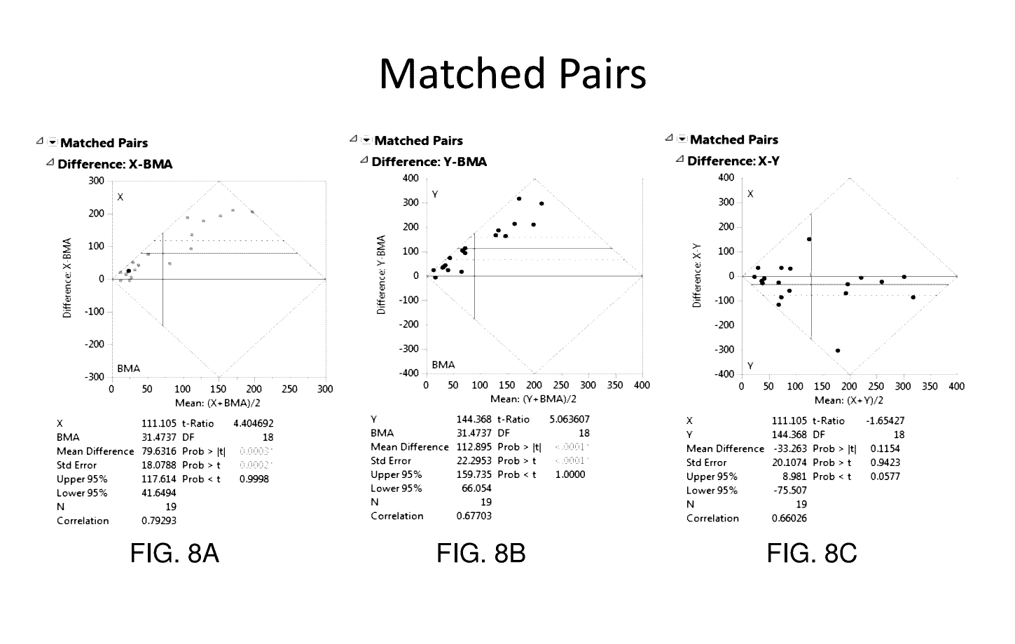

FIG. 9 is a representative bar graph showing platelet counts for BMA, BMC-X, and BMC-Y. Matched Pairs, paired T test Prob>t meaning that the difference between X & BMA and Y & BMA platelet counts is statistically significant, specifically there is an increase from BMA to X and BMA to Y. The different between X & Y is not statistically significant (prob<t). None of them (nor the differences between them) are normally distributed.

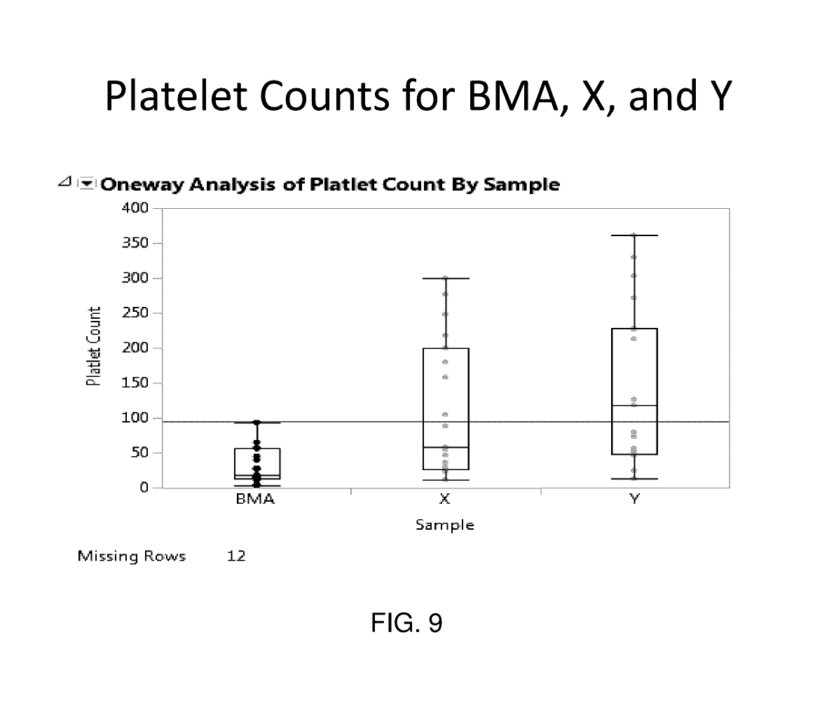

FIG. 10 is a representative bar graph showing nucleated cell count (NCC) of BMA, BMC-X, and BMC-Y groups.

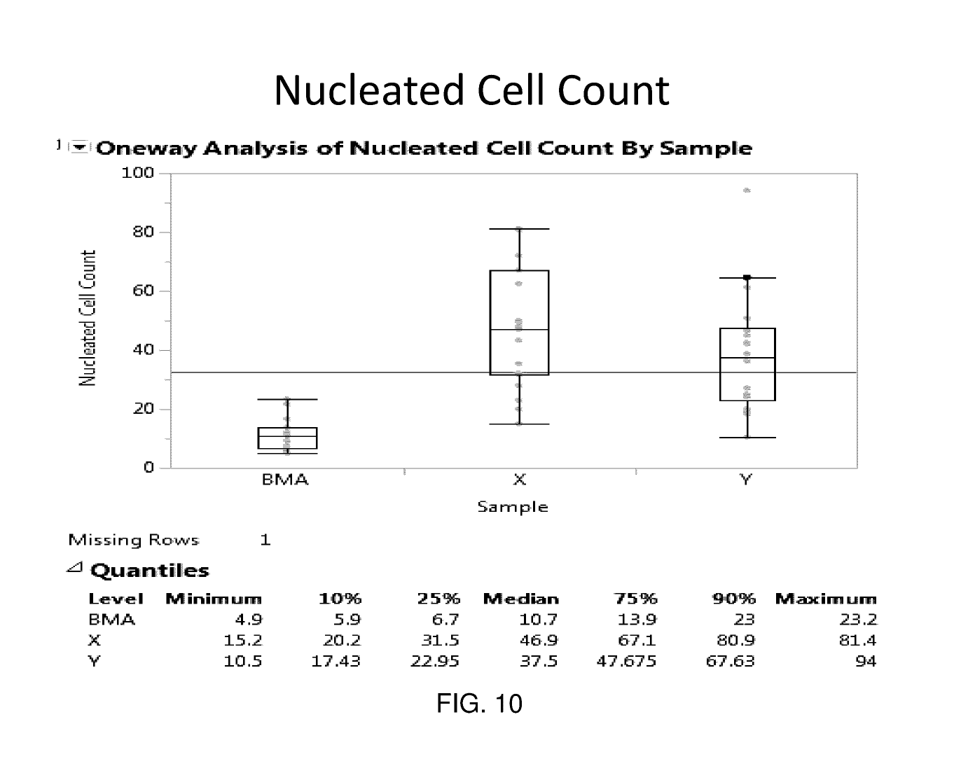

FIGS. 11A-11C are representative graphs showing NCC distributions of (FIG. 11A) BMA, (FIG. 11B) BMC-X, and (FIG. 11C) BMC-Y.

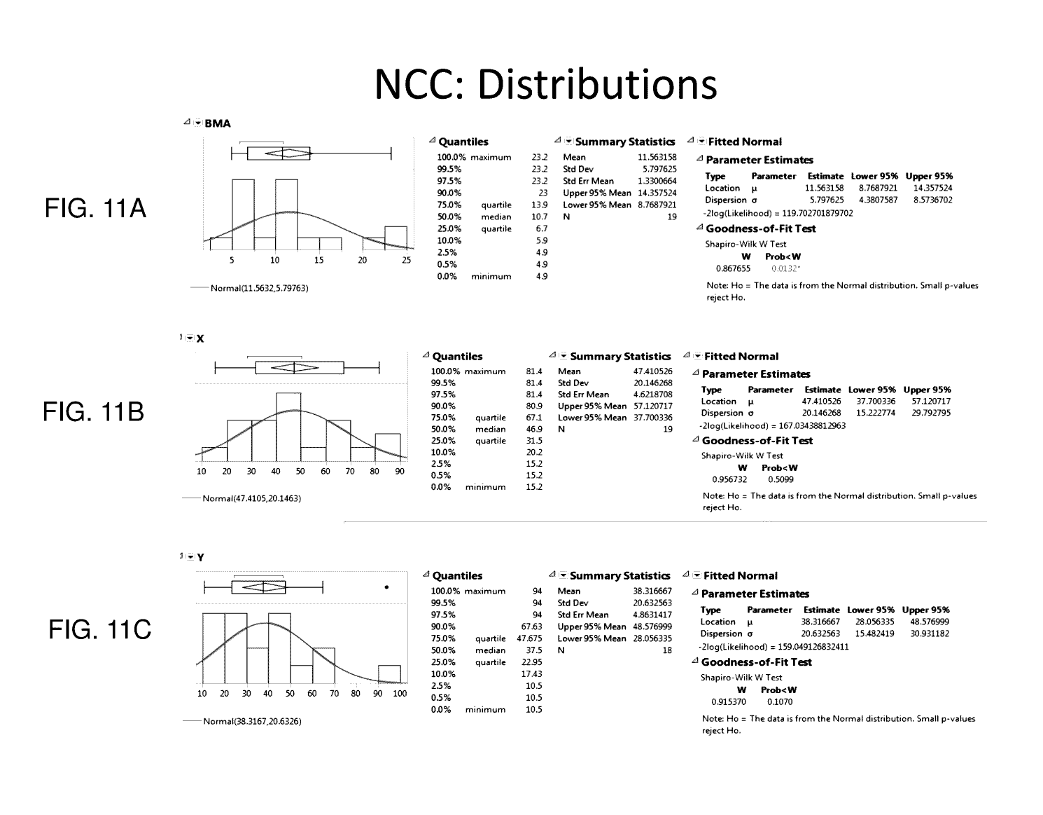

FIGS. 12A-12C are representative graphs showing difference in NCC distributions of (FIG. 12A) BMC-X vs BMA; (FIG. 12B) BMC-Y vs BMA; and (FIG. 12C) BMC-X vs BMC-Y.

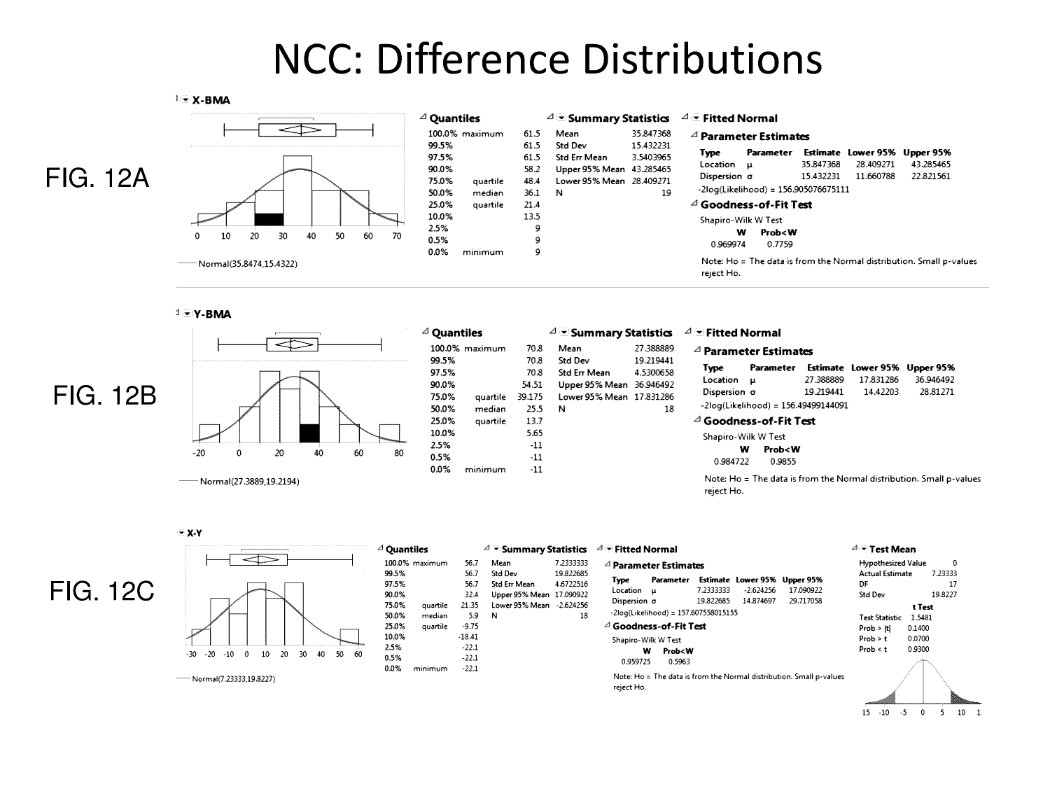

FIGS. 13A-13B are representative graphs showing comparison of NCC using a paired t-test in (FIG. 13A) BMC-X vs BMA and (FIG. 13B) BMC-Y vs BMA.

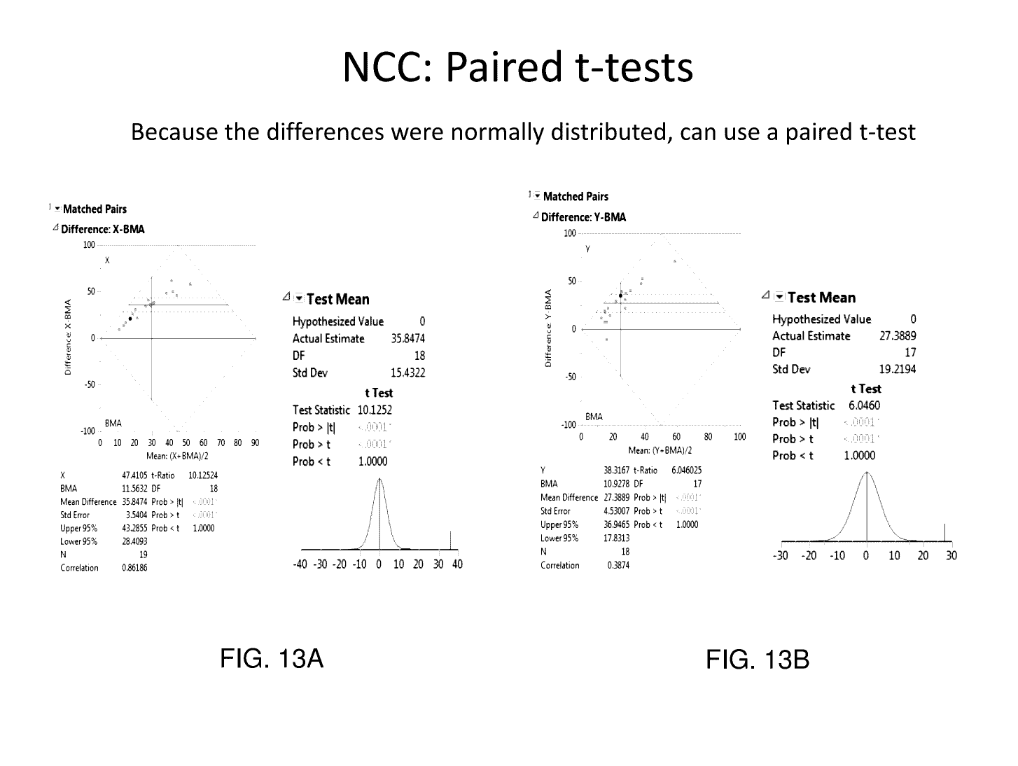

FIG. 14 is a representative graph showing comparison of NCC using a paired t-test in BMC-Y vs BMC-X.

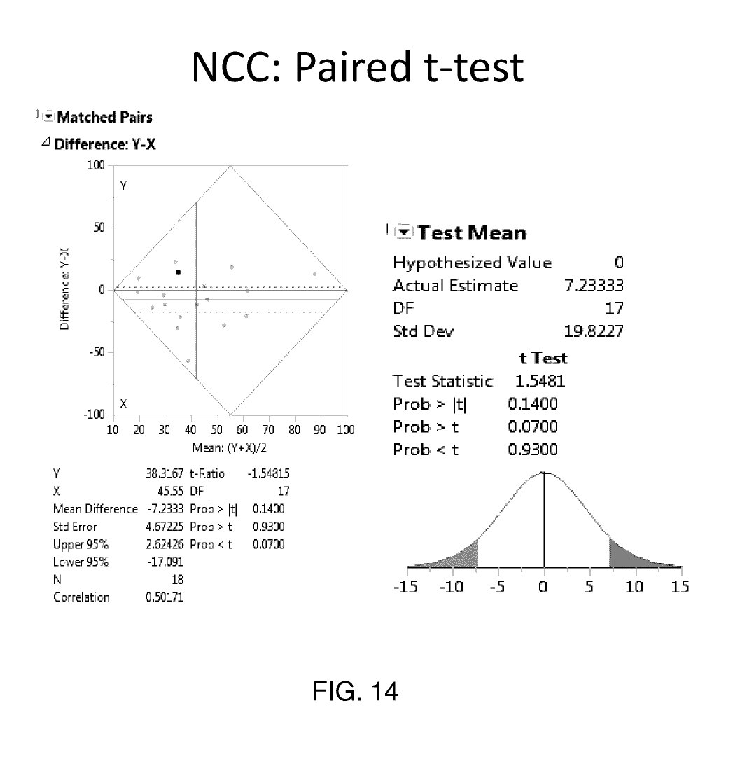

FIG. 15 is a representative graph showing colony count of BMA, BMC-X, and BMC-Y groups.

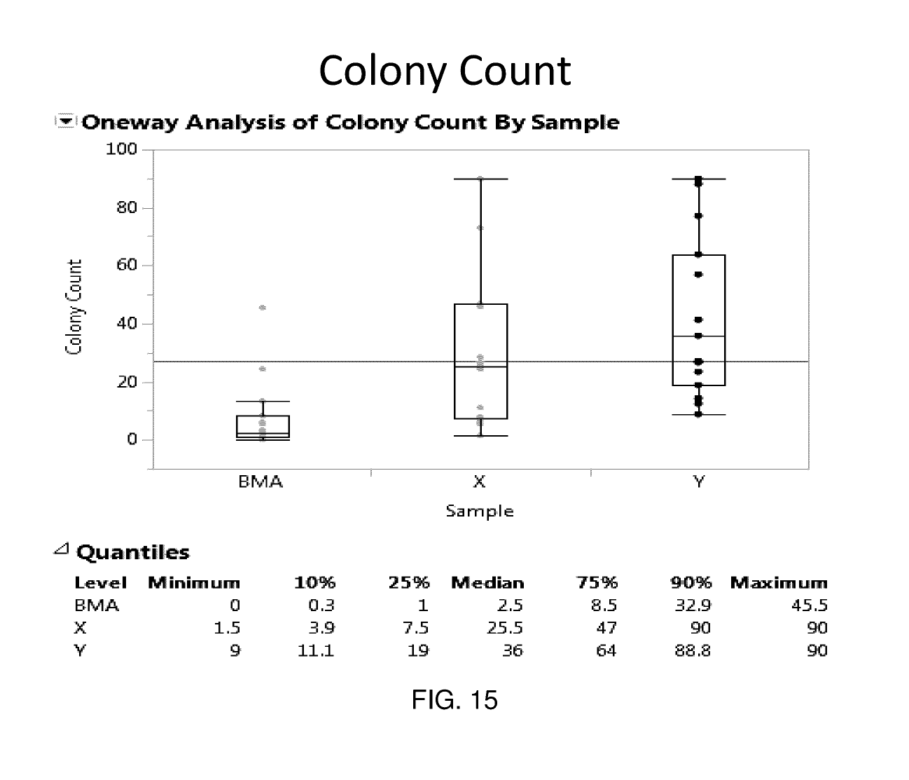

FIG. 16 is a representative graph showing colony count plate distribution. There is no statistically significant difference between the colony count duplicate plates.

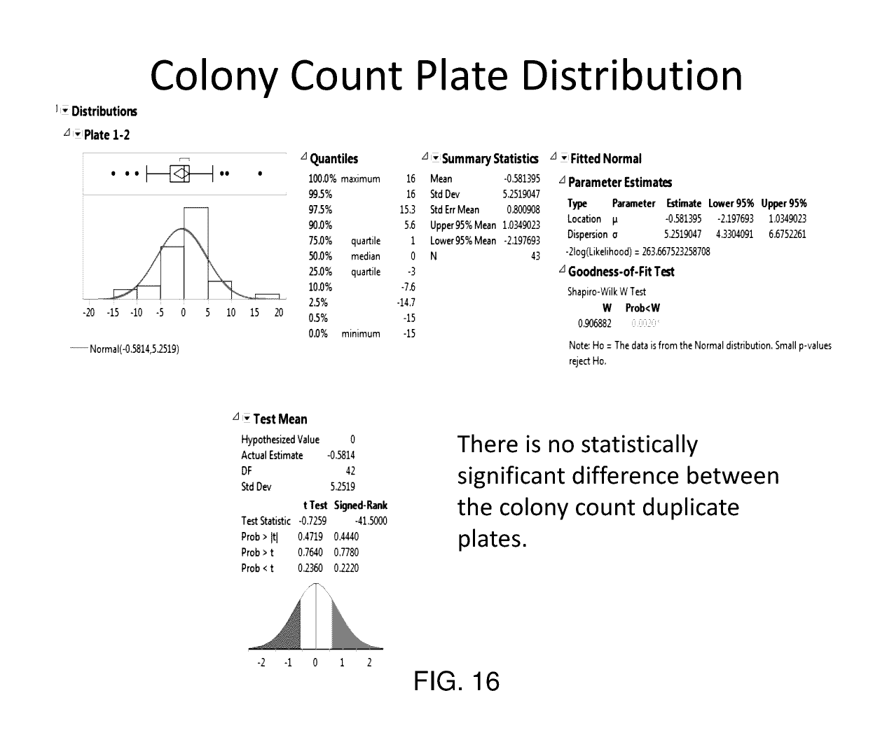

FIGS. 17A-17C are representative graphs showing colony count distributions of (FIG. 17A) BMA, (FIG. 17B) BMC-X, and (FIG. 17C) BMC-Y.

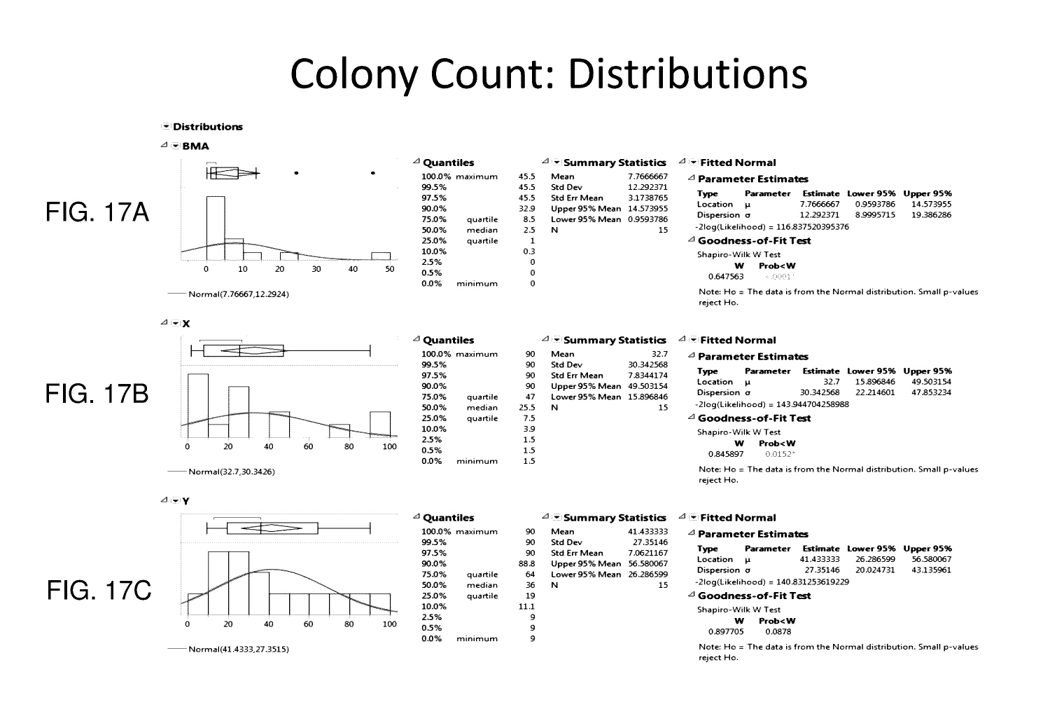

FIGS. 18A-18B are representative graphs showing comparison of colony count difference in distribution of (FIG. 18A) BMC-X vs BMA; and (FIG. 18B) BMC-Y vs BMA. Distribution of the differences are normal, therefore can use paired t-test. Both X and Y colony counts are statistically greater than BMA.

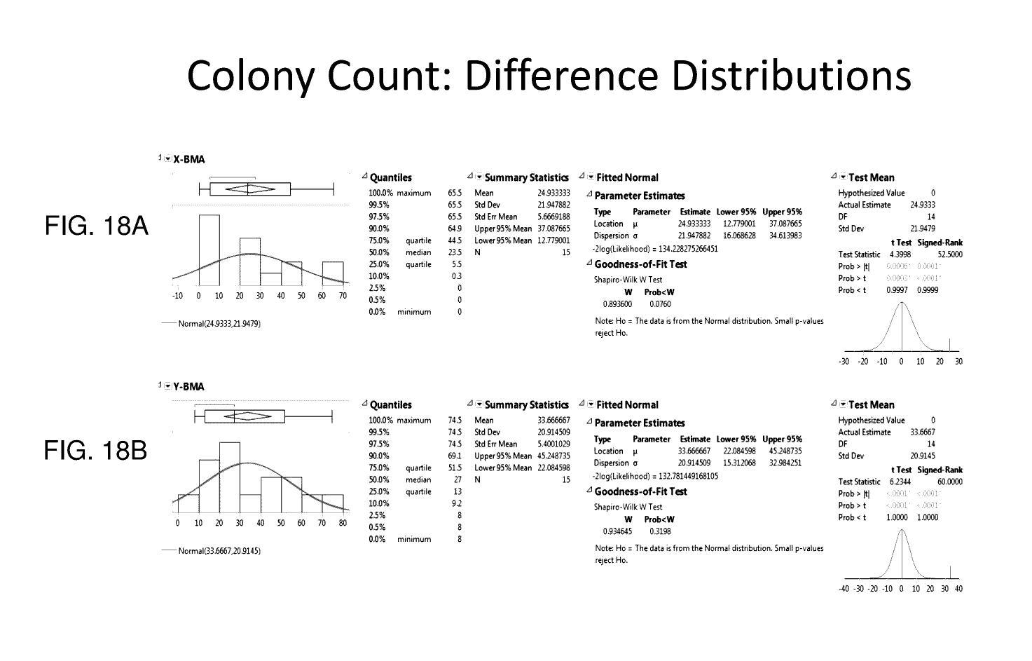

FIGS. 19A-19B are representative graphs showing comparison of colony count difference in distribution of (FIG. 19A) BMC-X vs BMC-Y; and (FIG. 19B) BMC-Y vs BMC-X. Distribution of the differences are normal, therefore can use paired t-test. Y colony count is significantly greater than X colony count.

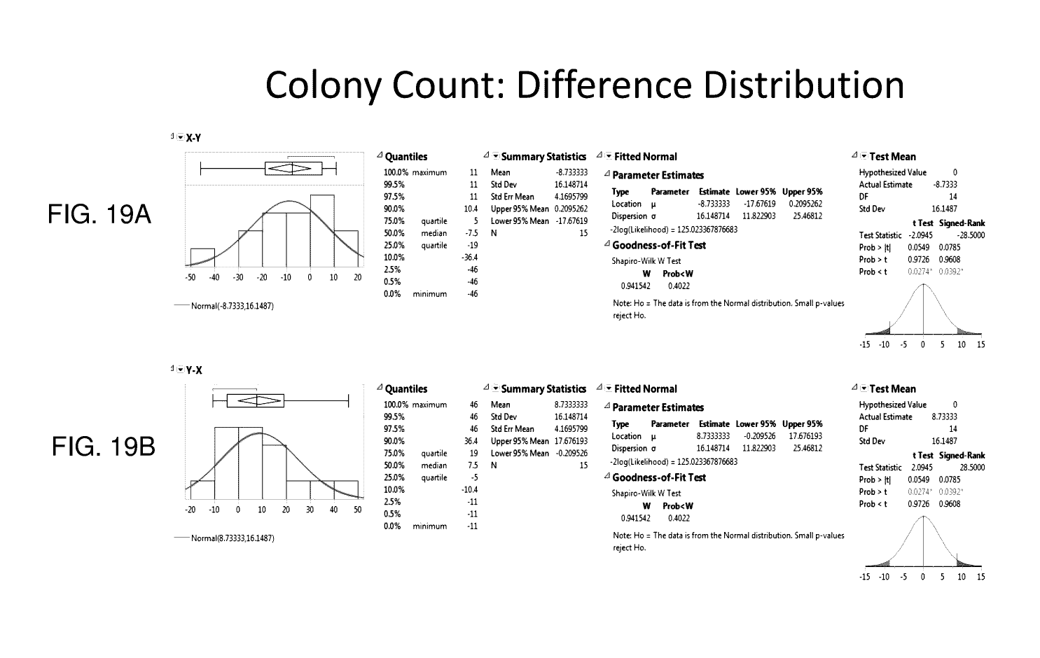

FIG. 20 is a representative graph showing observed frequency of IL-1β in BMA, BMC-X, and BMC-Y.

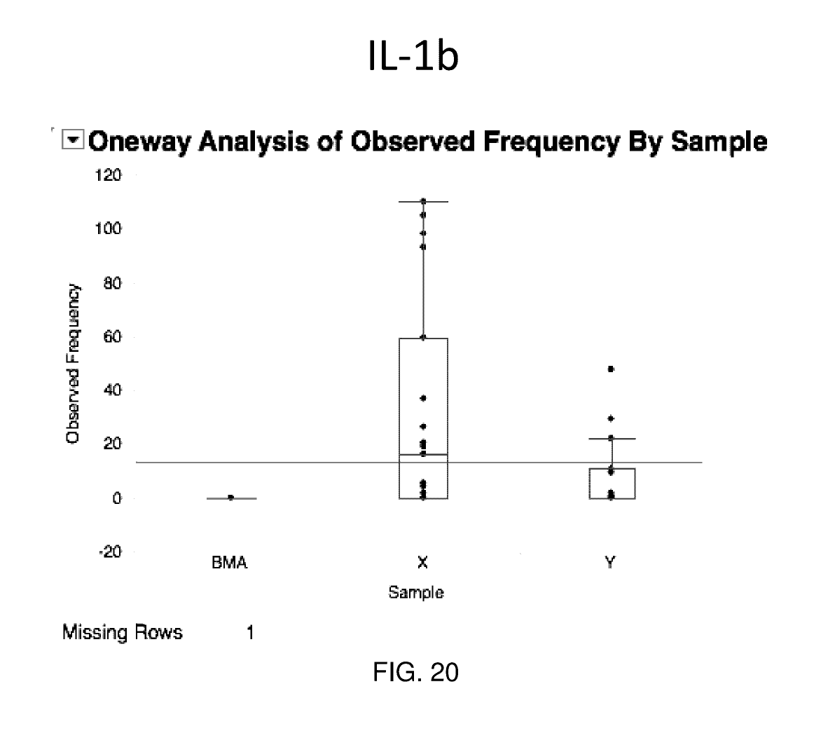

FIGS. 21A-21C are representative graphs showing IL-1β distributions in (FIG. 21A) BMC-Y, (FIG. 21B) BMC-X, and (FIG. 21C) BMC-X/Y. All observed concentrations for IL-1b in BMA were 0.

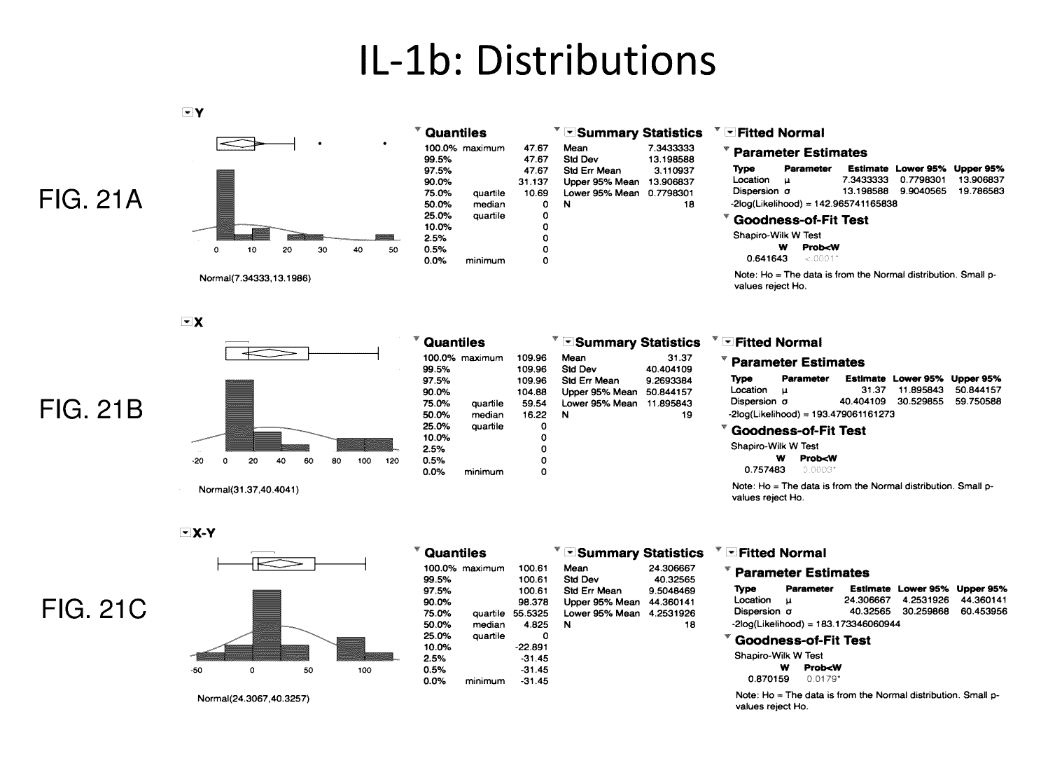

FIG. 22 is a representative graph showing comparison of IL-1β of BMC-X vs BMC-Y. Distribution of differences between X and Y is not normally distributed, must use Wilcoxin Signed Rank Test.

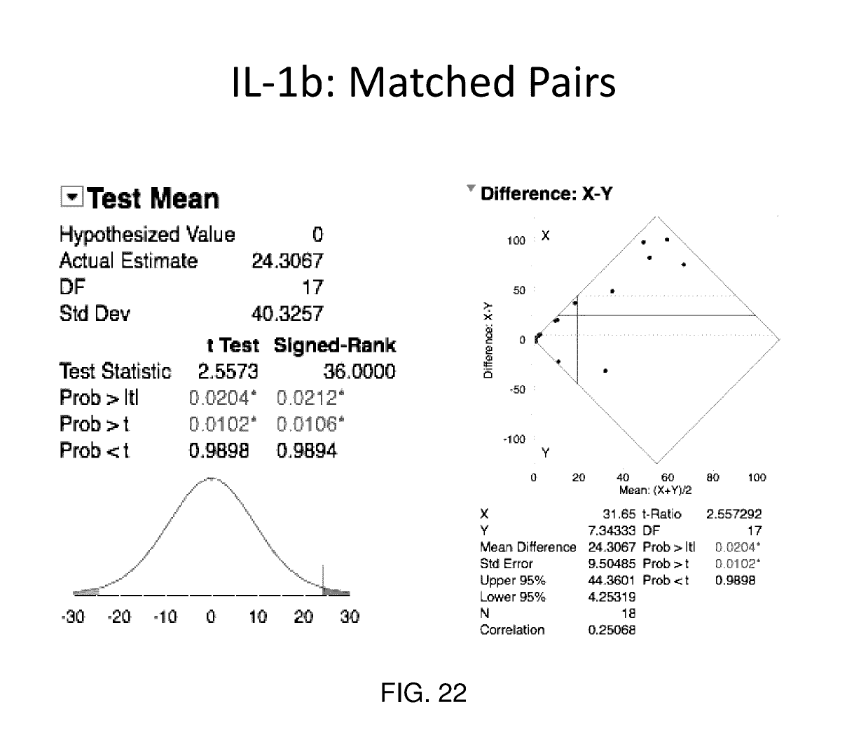

FIG. 23 is a representative graph showing observed frequency of IL-1β in BMA, BMC-X, and BMC-Y. IL-1b concentration observed in BMC-X is significantly greater than the concentration observed in BMC-Y.

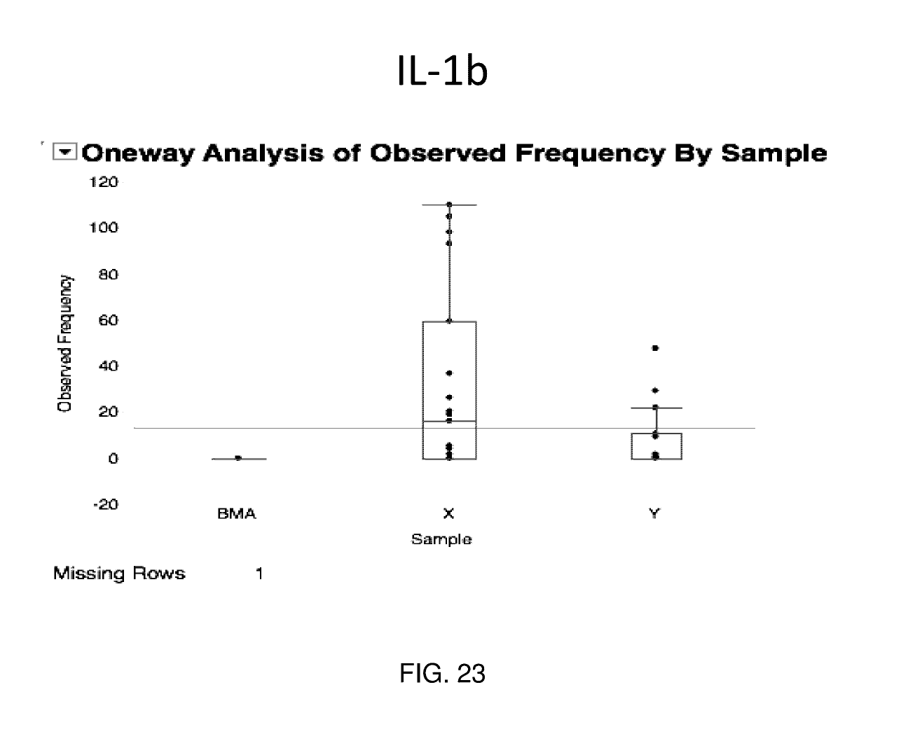

FIG. 24 is a representative graph showing observed frequency of IL-ra n BMA, PRP, whole blood (WB), BMC-X, and BMC-Y.

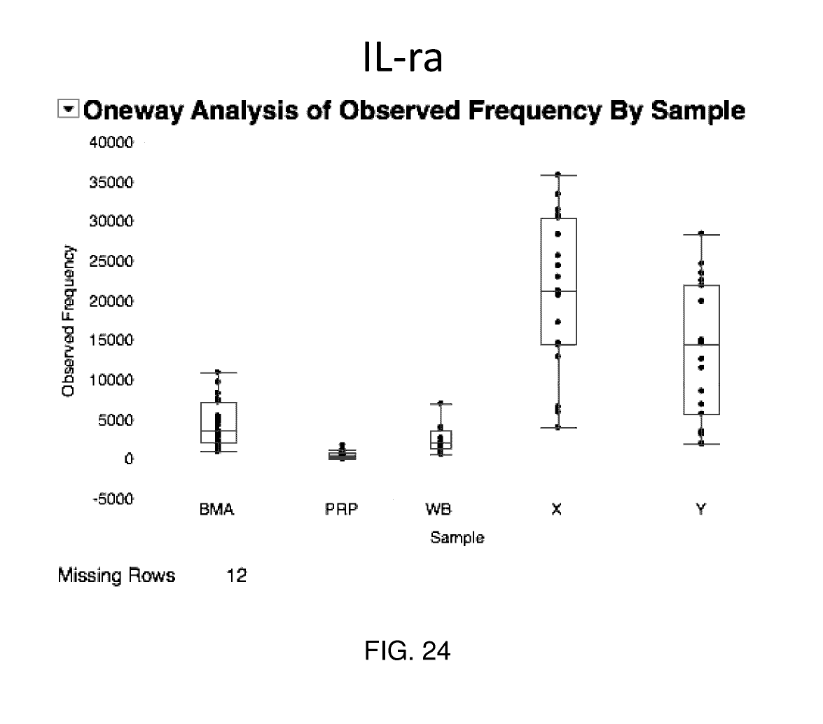

FIGS. 25A-25C are representative graphs showing IL-ra distributions for (FIG. 25A) BMA, (FIG. 25B) BMC-X, and (FIG. 25C) BMC-Y.

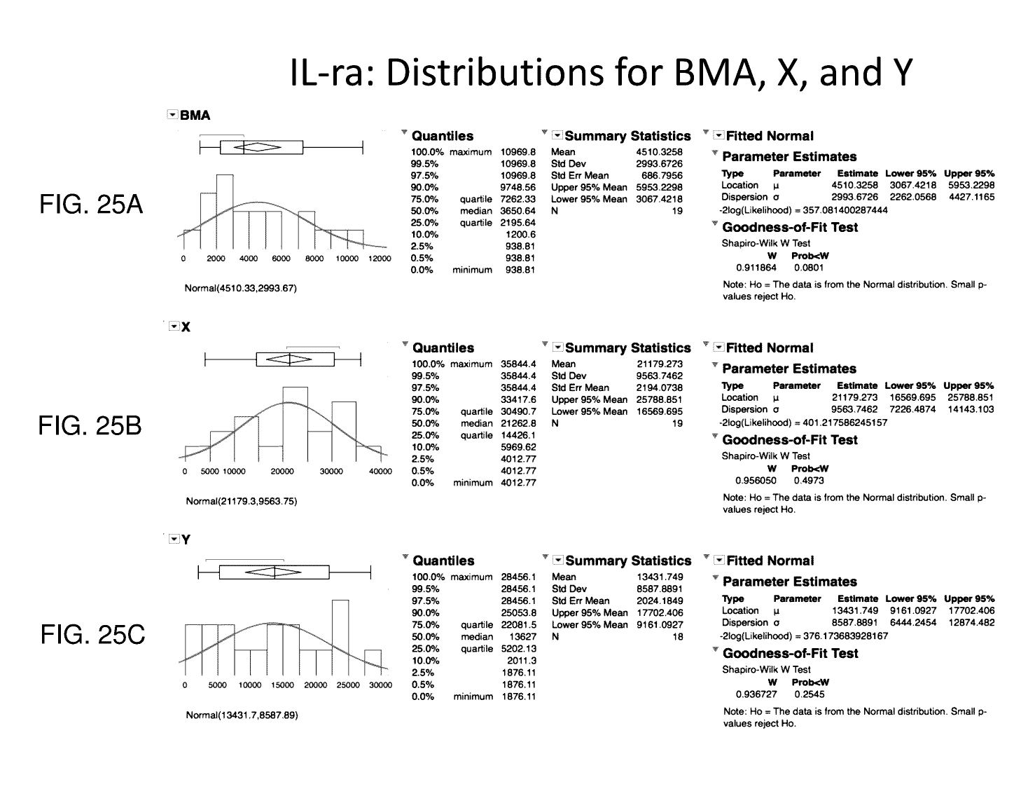

FIGS. 26A-26C are representative graphs showing comparison of IL-ra in difference distributions of (FIG. 26A) BMC-X vs BMA; (FIG. 26B) BMC-Y vs BMA and (FIG. 26C) BMC-X vs BMC-Y.

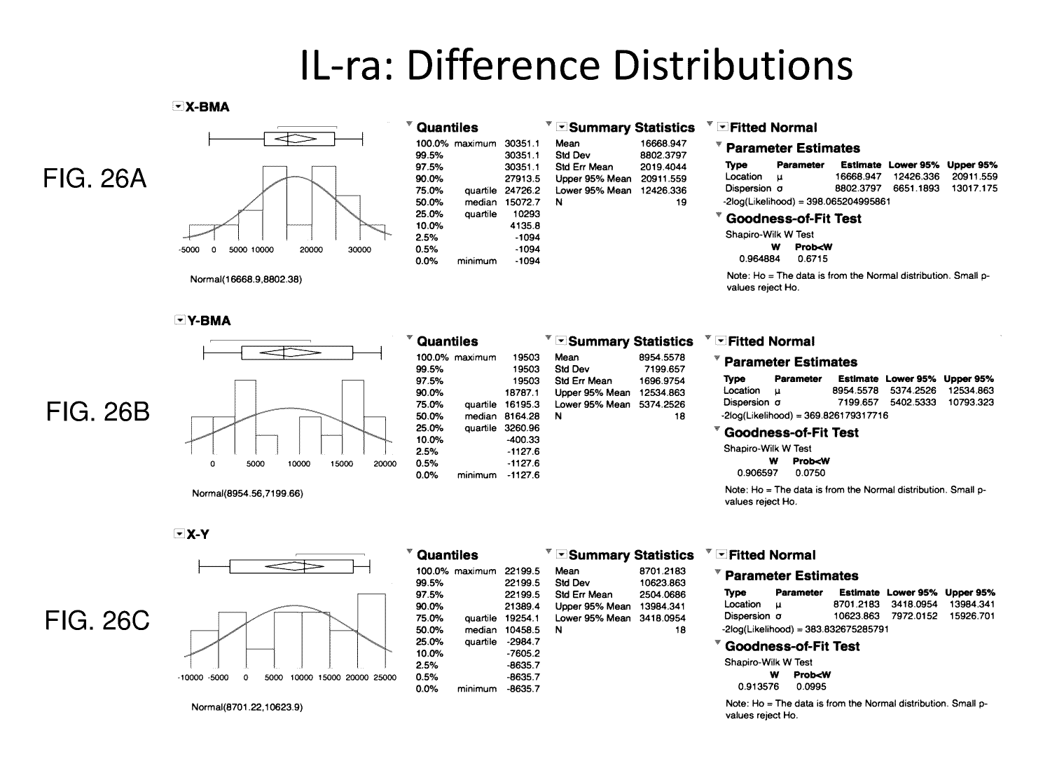

FIGS. 27A-27C are representative graphs showing comparison of IL-ra in difference distributions of (FIG. 27A) BMC-X vs BMA; (FIG. 27B) BMC-Y vs BMA and (FIG. 27C) BMC-X vs BMC-Y using paired t-tests. Because the differences are normally distributed, can use paired t-test. All are significantly different from the other.

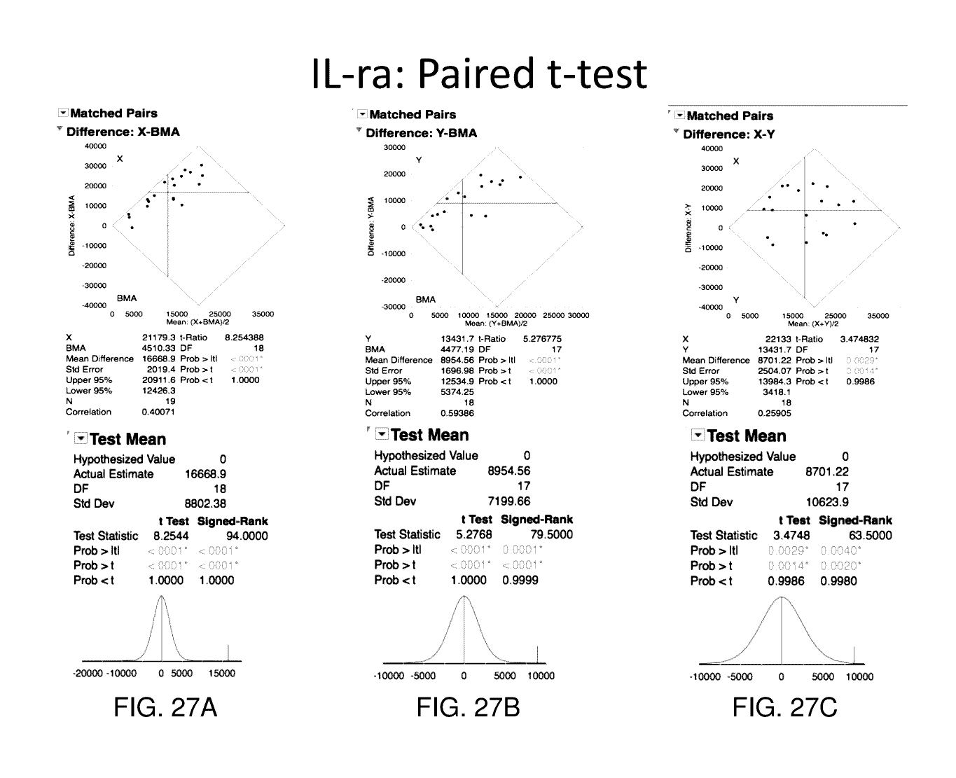

FIGS. 28A-28C are representative graphs showing comparison of IL-ra in difference distributions for (FIG. 28A) PRP, (FIG. 28B) whole blood, and (FIG. 28C) WB vs PRP.

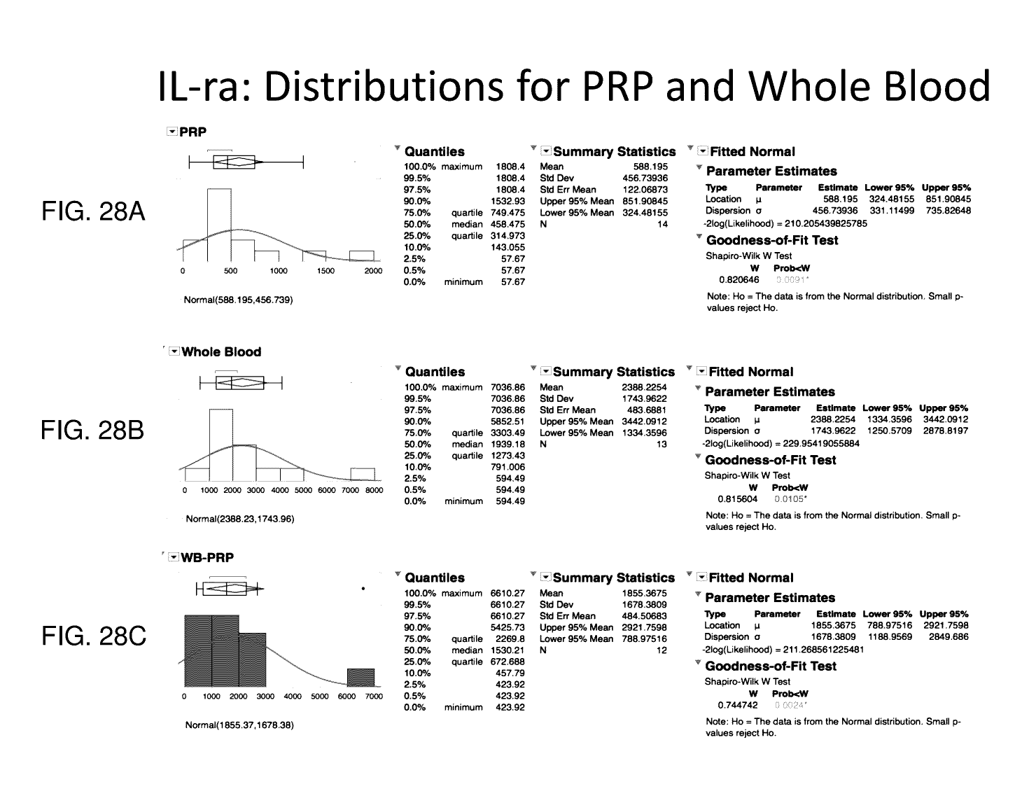

FIG. 29 is a representative graph showing IL-ra difference distribution for WB vs PRP using Wilcoxon Signed Rank test. Because the difference is not normally distributed, must use Wilcoxon Signed Rank Test. There is a significant difference between whole blood and PRP.

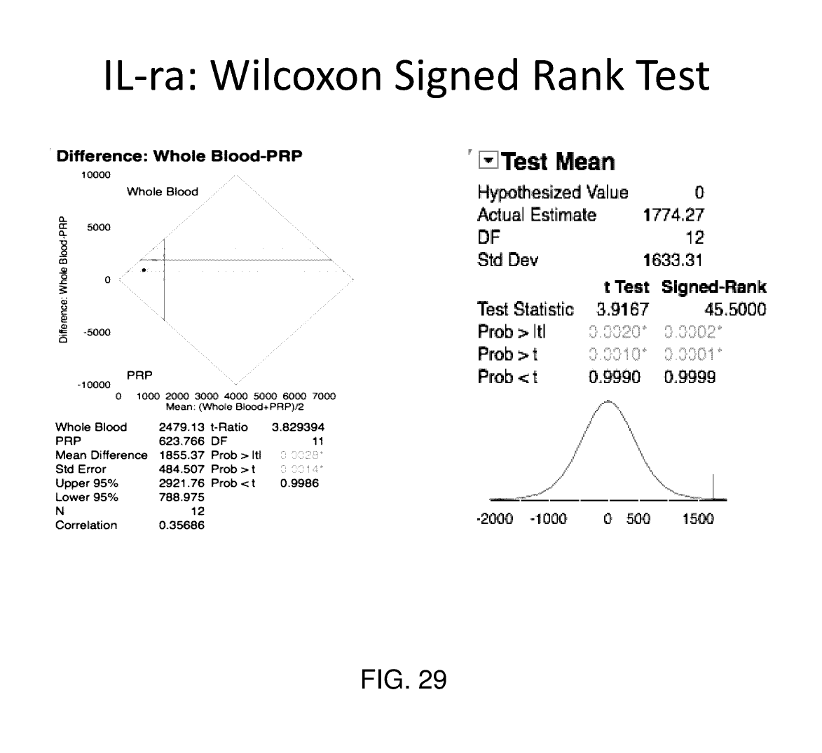

FIGS. 30A-30B are representative graphs showing comparison of IL-ra in difference distributions for (FIG. 30A) BMC-X vs PRP and (FIG. 30B) BMC-Y vs PRP.

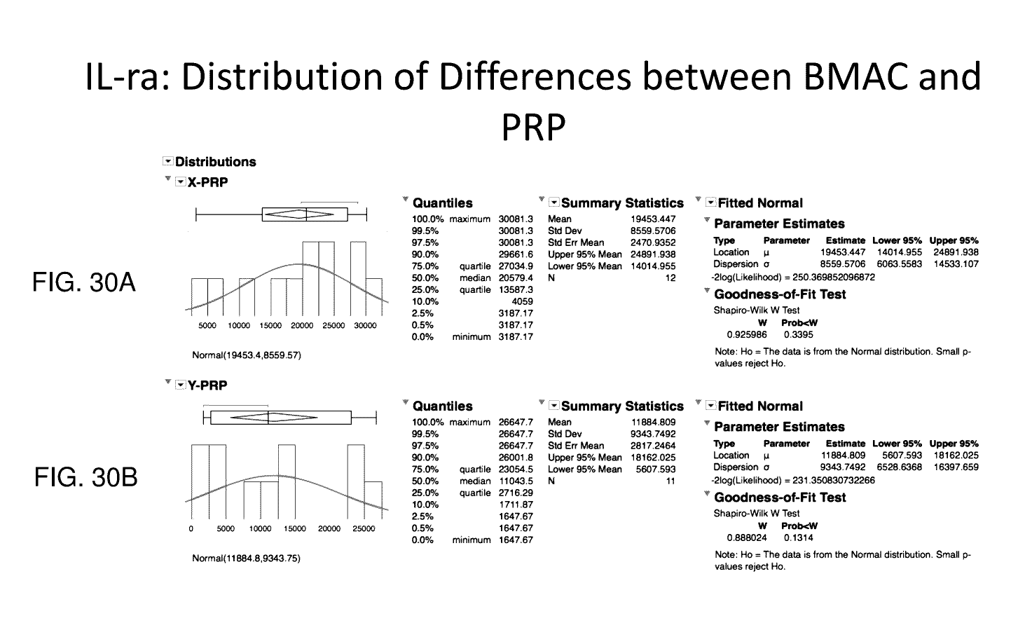

FIGS. 31A-31B are representative graphs showing comparison of IL-ra in difference distributions for (FIG. 31A) BMC-X vs PRP and (FIG. 31B) BMC-Y vs PRP using paired t-test.

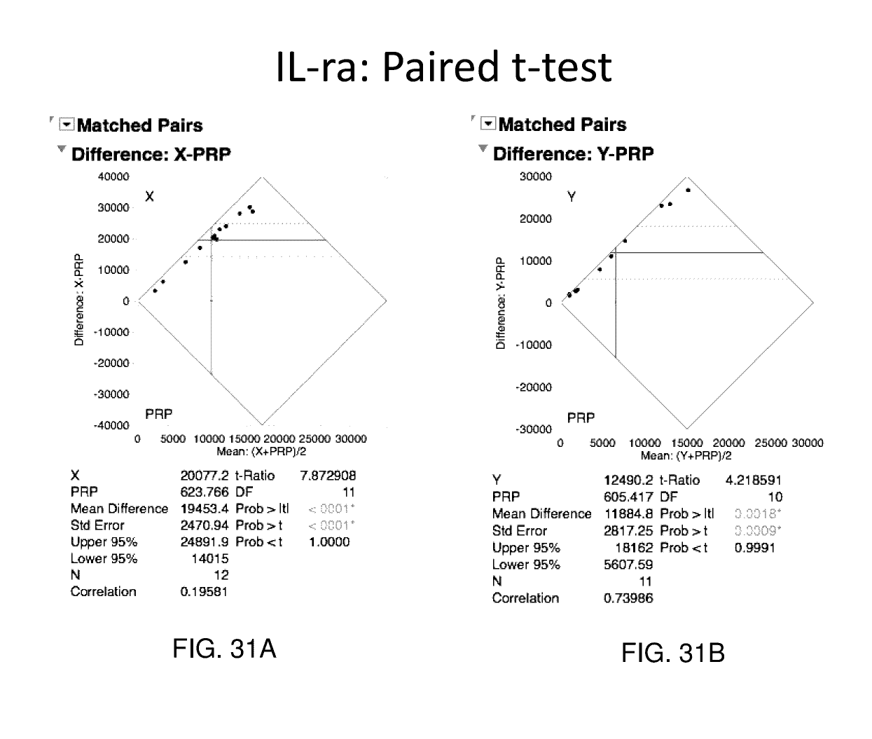

FIG. 32 is a representative graph showing observed frequency of IL-ra in BMA, PRP, WB, BMC-X and BMC-Y. Based on the paired t-tests and Wilcoxon Signed Rank Test the concentration of IL-ra in: BMC-X is significantly greater than BMA; BMC-Y is significantly greater than BMA; BMC-X is significantly greater than BMC-Y; Whole blood is significantly greater than PRP; BMC-X is significantly greater than PRP; BMC-Y is significantly greater than PRP.

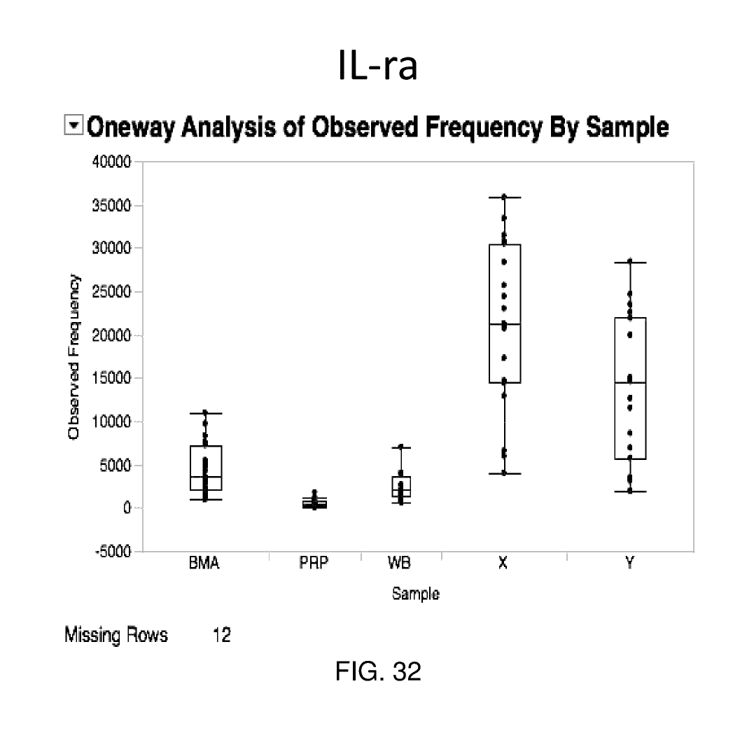

FIG. 33 is a representative graph showing observed frequency of IL-8 in BMA, BMC-X and BMC-Y.

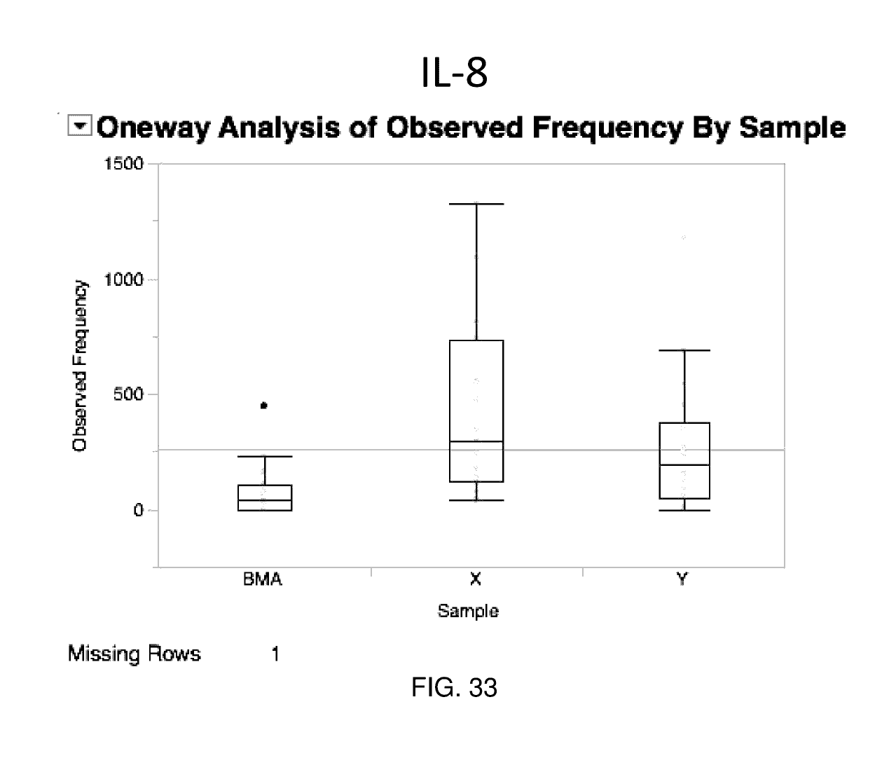

FIGS. 34A-34C are representative graphs showing IL-8 distributions for (FIG. 34A) BMA, (FIG. 34B) BMC-X, and (FIG. 34C) BMC-Y.

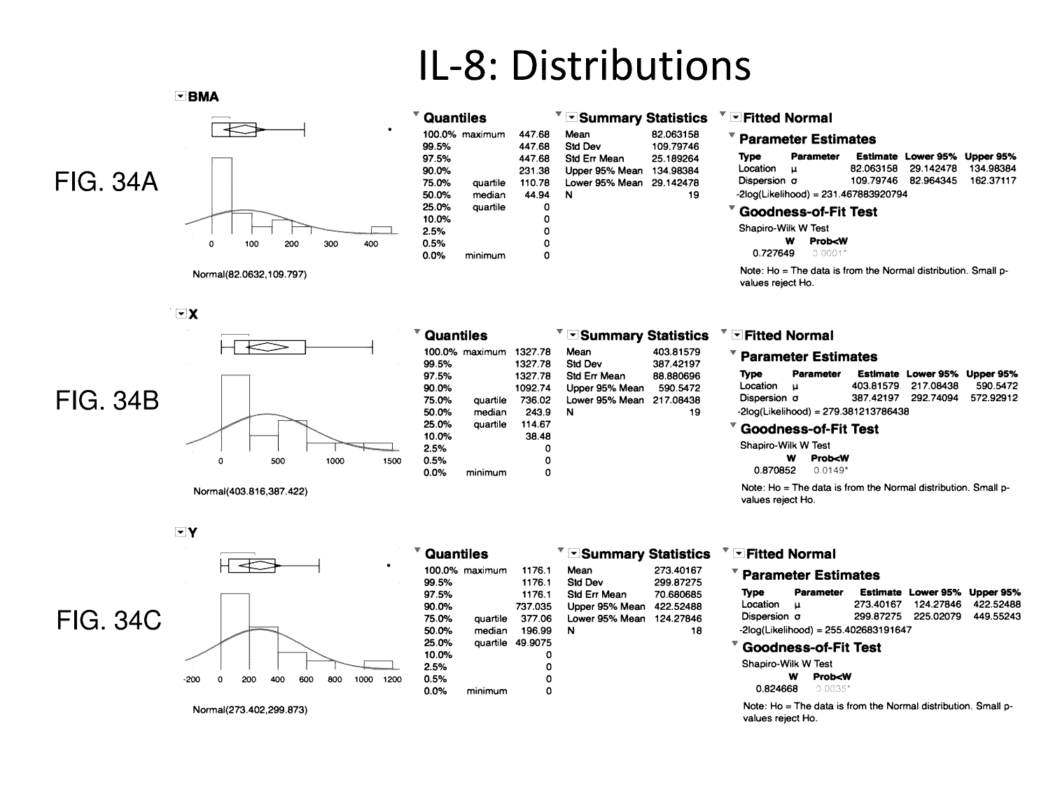

FIGS. 35A-35C are representative graphs showing IL-8 difference in distributions for (FIG. 35A) BMC-X vs BMA; (FIG. 35B) BMC-Y vs BMA and (FIG. 35C) BMC-X vs BMC-Y. Must use Wilcoxon Signed Rank for BMC-X vs BMA and BMC-Y vs BMA, can use paired t-test for BMC-X vs BMC-Y.

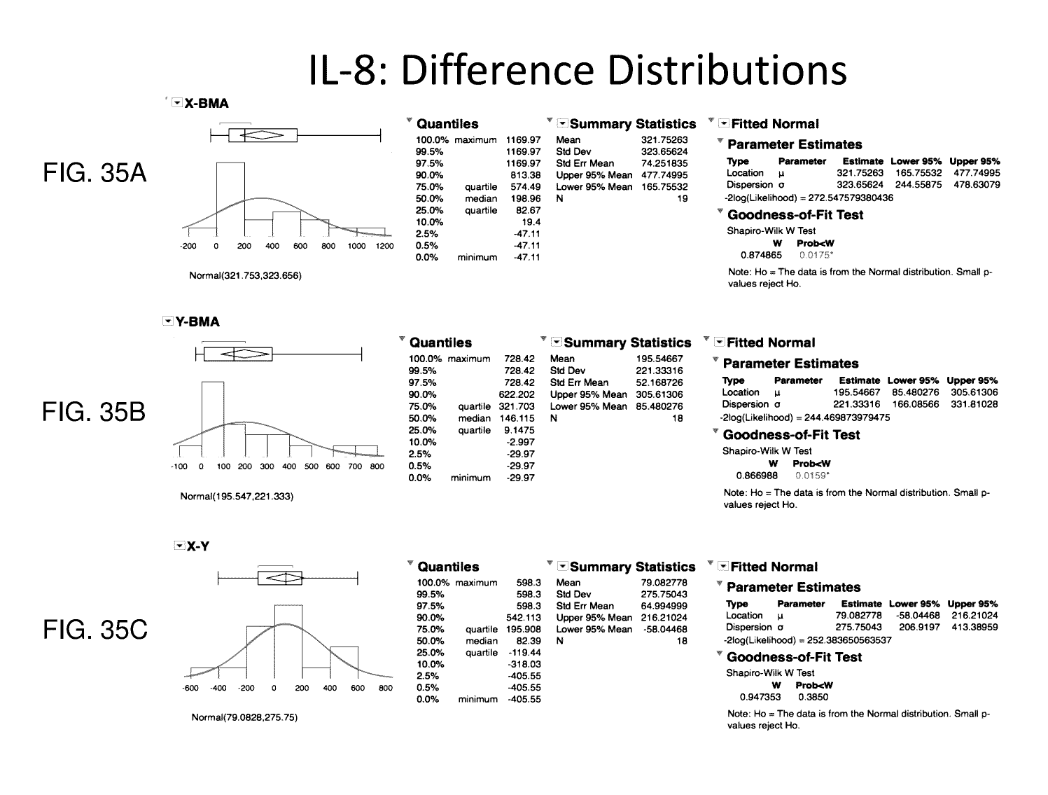

FIGS. 36A-36C are representative graphs showing IL-8 difference in distributions for (FIG. 36A) BMC-X vs BMA; (FIG. 36B) BMC-Y vs BMA and (FIG. 36C) BMC-X vs BMC-Y with matched pairs.

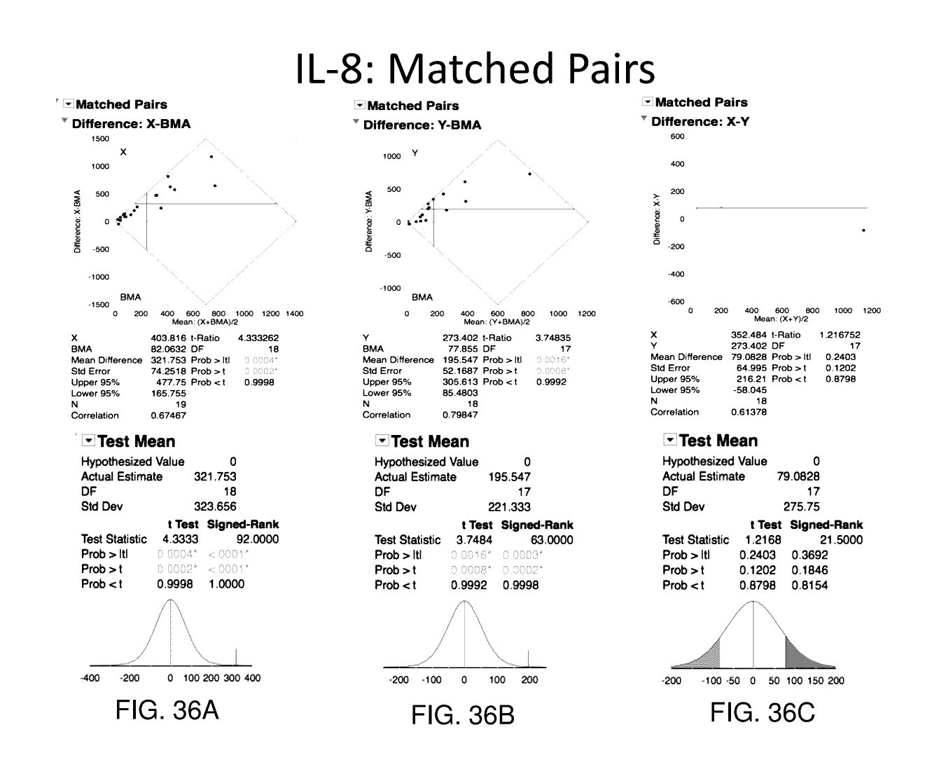

FIG. 37 is a representative graph showing observed frequency of IL-8 in BMA, BMC-X and BMC-Y. Based on the Wilcoxon Rank Sum Test, there is a significant difference between BMC-X and BMA as well as BMC-Y and BMA. Based on the paired t-test, there is no significant difference between the mean concentration of IL-8 in BMC-X compared to that in BMC-Y.

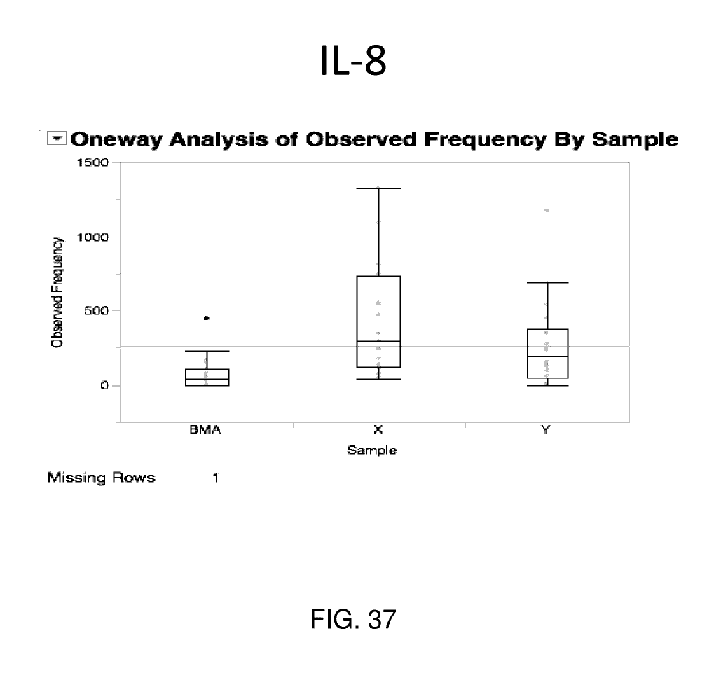

FIG. 38 is a representative graph showing observed frequency of PDGF in BMA, PRP, WB, BMC-X, and BMC-Y.

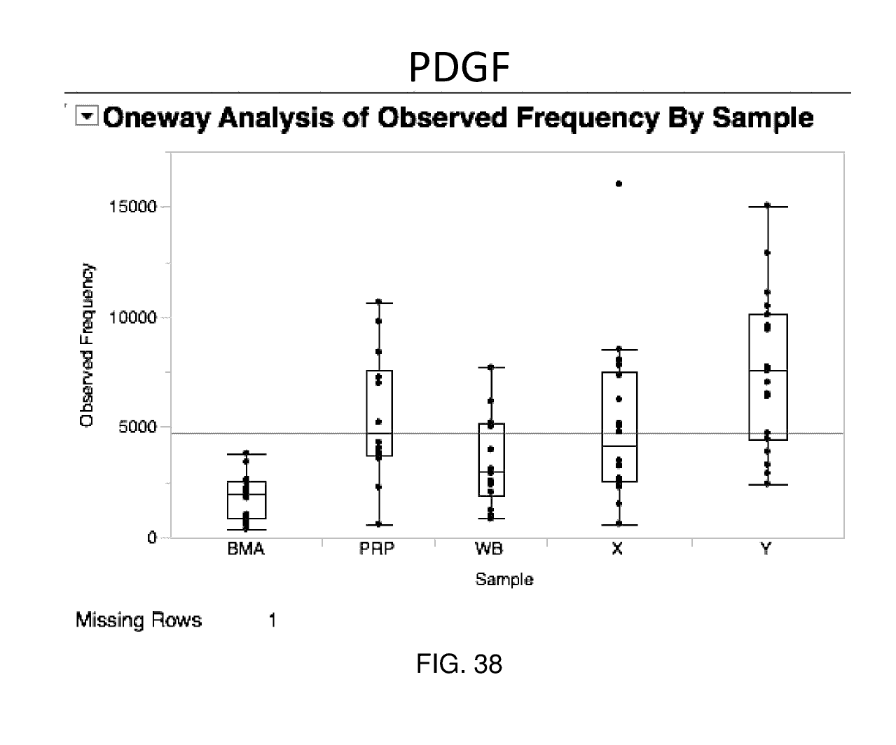

FIGS. 39A-39C are representative graphs showing PDGF distributions for (FIG. 39A) BMA, (FIG. 39B) BMC-X, and (FIG. 39C) BMC-Y.

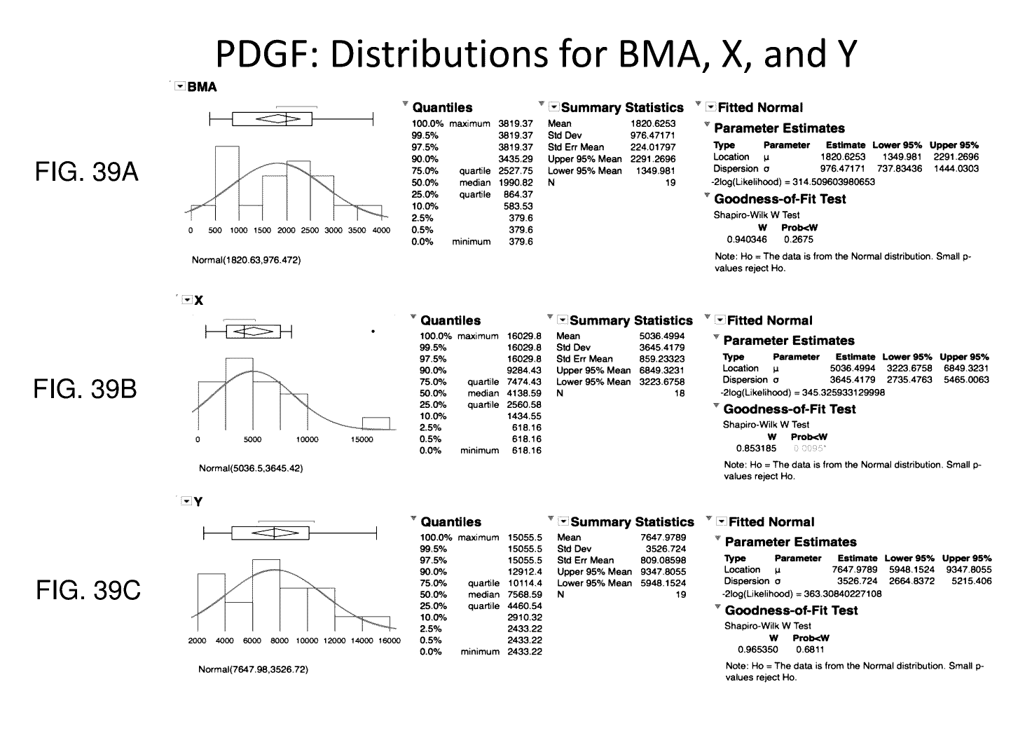

FIGS. 40A-40C are representative graphs showing comparison of PDGF difference in distributions for (FIG. 40A) BMC-X vs BMA; (FIG. 40B) BMC-Y vs BMA and (FIG. 40C) BMC-X vs BMC-Y.

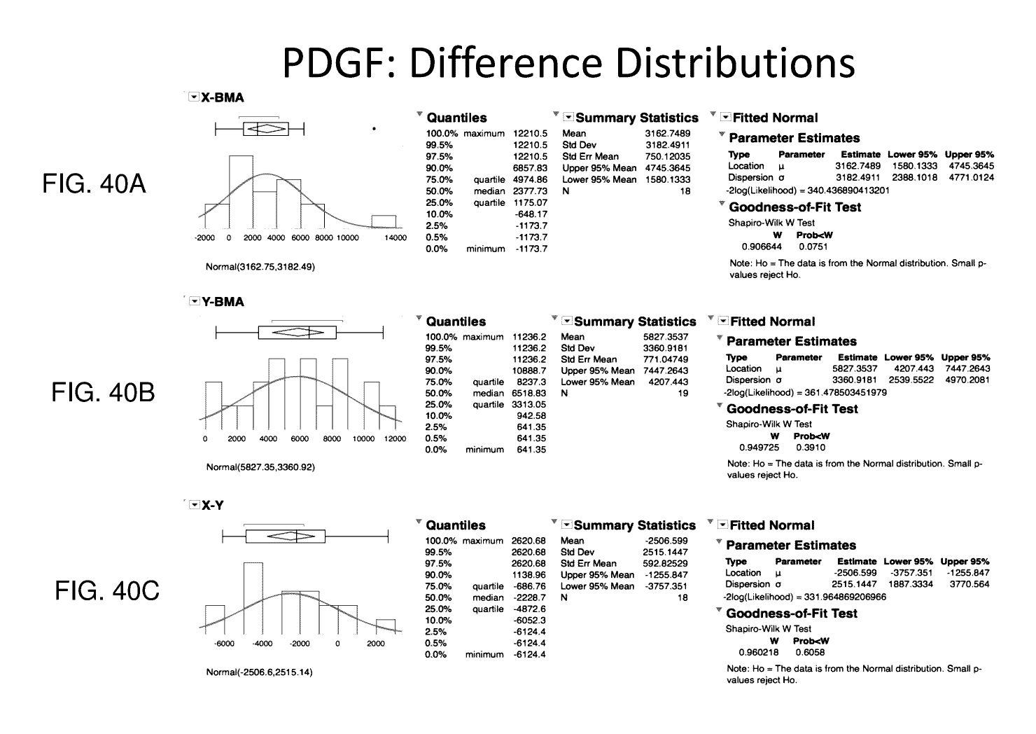

FIGS. 41A-41C are representative graphs showing PDGF distributions for (FIG. 41A) PRP and (FIG. 41B) whole blood; and (FIG. 41C) comparison of PDGF distribution for WB vs PRP.

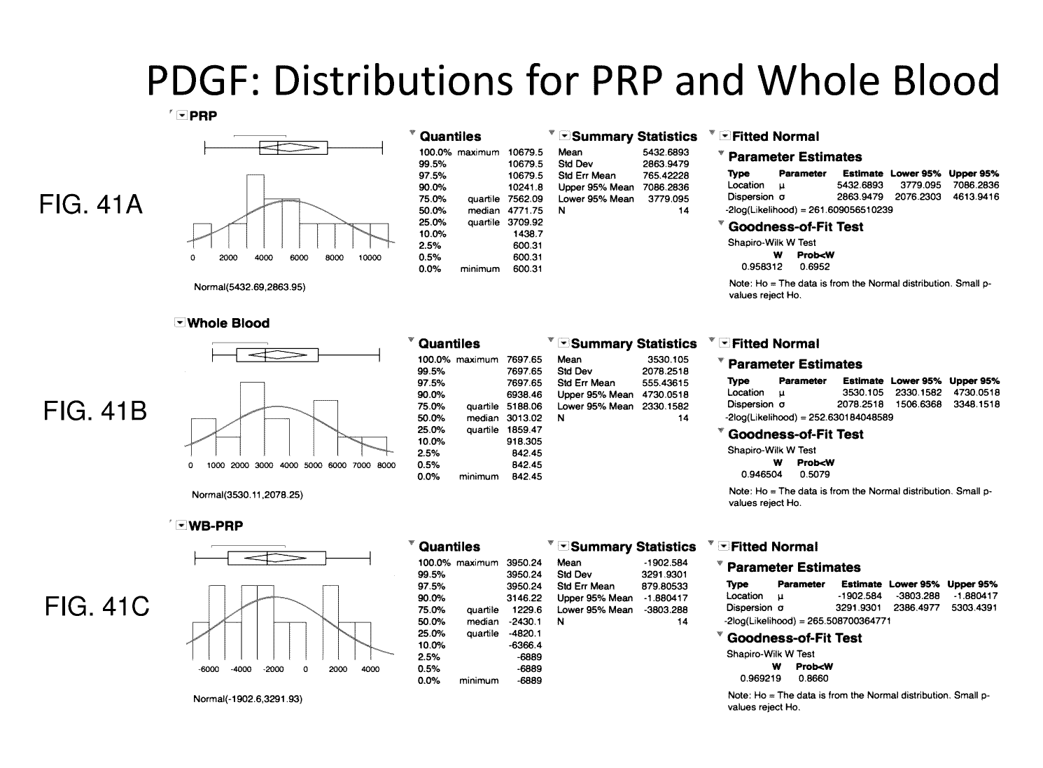

FIGS. 42A-42D are representative graphs showing comparison of PDGF for (FIG. 42A) BMC-X vs BMA; (FIG. 42B) BMC-Y vs BMA, (FIG. 42C) BMC-X vs BMC-Y and (D) PRP vs whole blood.

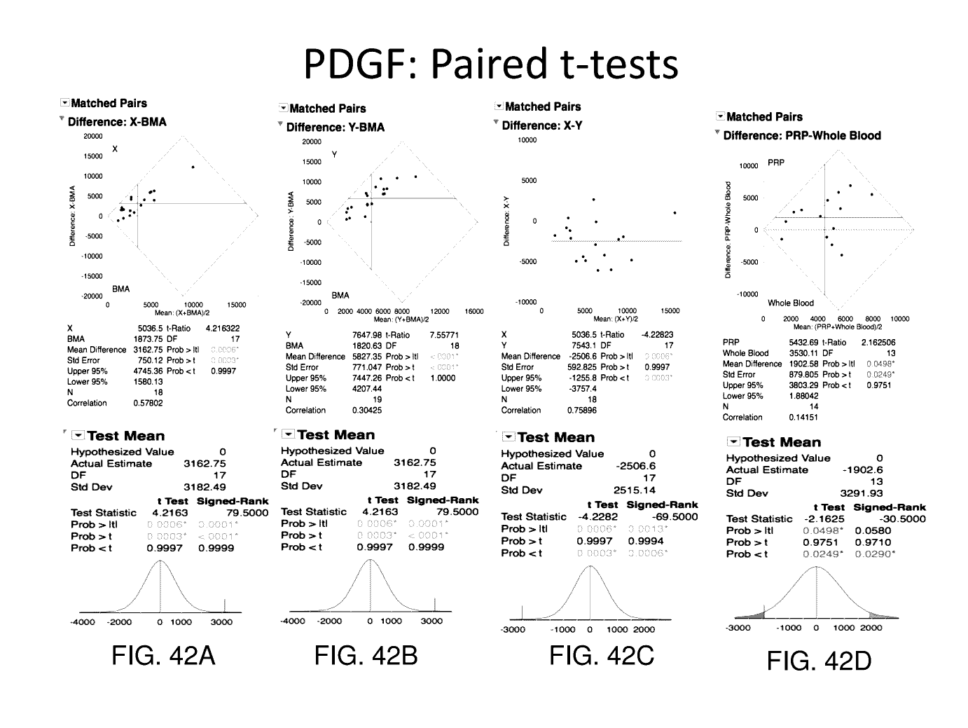

FIGS. 43A-43B are representative graphs showing PDGF distributions of difference between (FIG. 43A) BMC-X and PRP; and (FIG. 43B) BMC-Y and PRP.

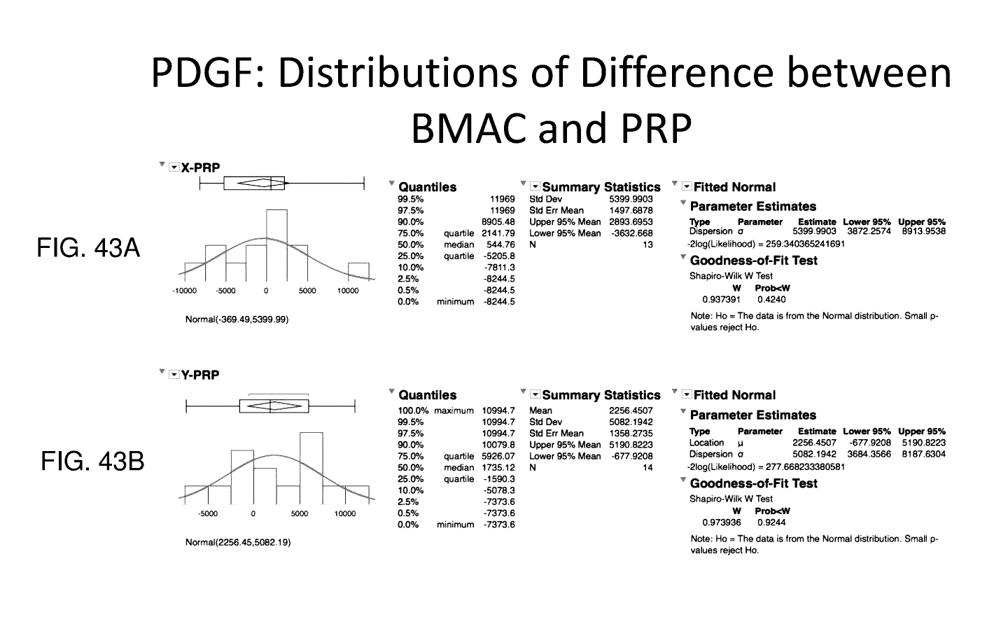

FIGS. 44A-44B are representative graphs showing PDGF distributions of difference between (FIG. 44A) BMC-X and PRP; and (FIG. 44B) BMC-Y and PRP using paired t-test.

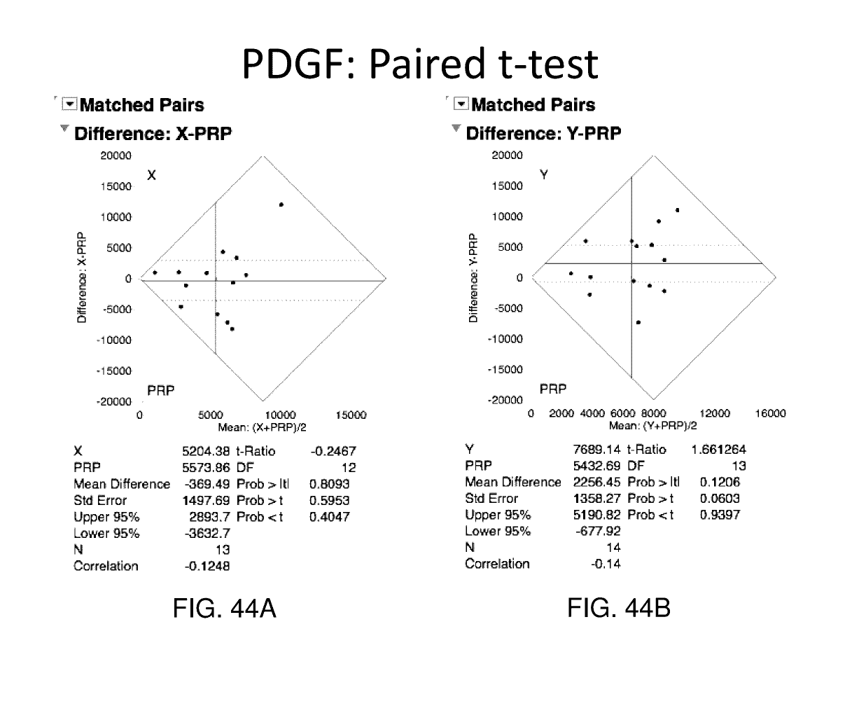

FIG. 45 is a representative graph showing observed frequency of PDGF in BMA, PRP, WB, BMC-X and BMC-Y. Based on the paired t-tests the concentration of PDGF in: BMC-X is significantly greater than BMA; BMC-Y is significantly greater than BMA; BMC-Y is significantly greater than BMC-X; PRP is significantly greater than Whole Blood. There is no significant difference between either BMC-X or BMC-Y and PRP.

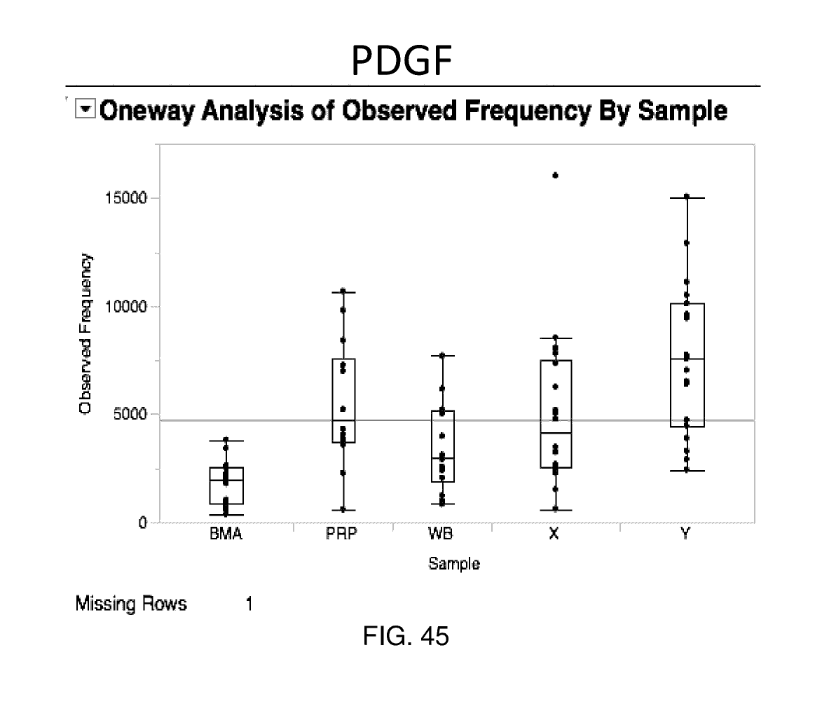

FIG. 46 is a representative graph showing correlation of platelet count and PDGF. Correlation coefficient between platelet count and PDGF is 0.668.

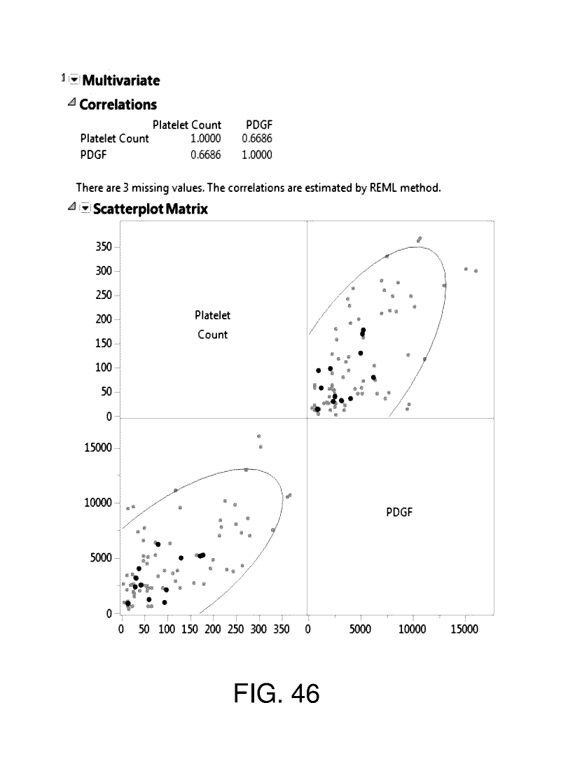

FIGS. 47A-47B are representative graphs showing correlation of IL-1b and neutrophil count in (FIG. 47A) BMC-X and (FIG. 47B) BMC-Y. Correlation Coefficient is calculated for BMC-X and BMC-Y (the only samples with IL-1b measurements). Neutrophil count is based on NCC and Neutrophil % estimation from smear. Correlation between IL-1b and neutrophil count is 0.52 when the samples marked <OOR samples as 0 are included and 0.47 when these samples are excluded.

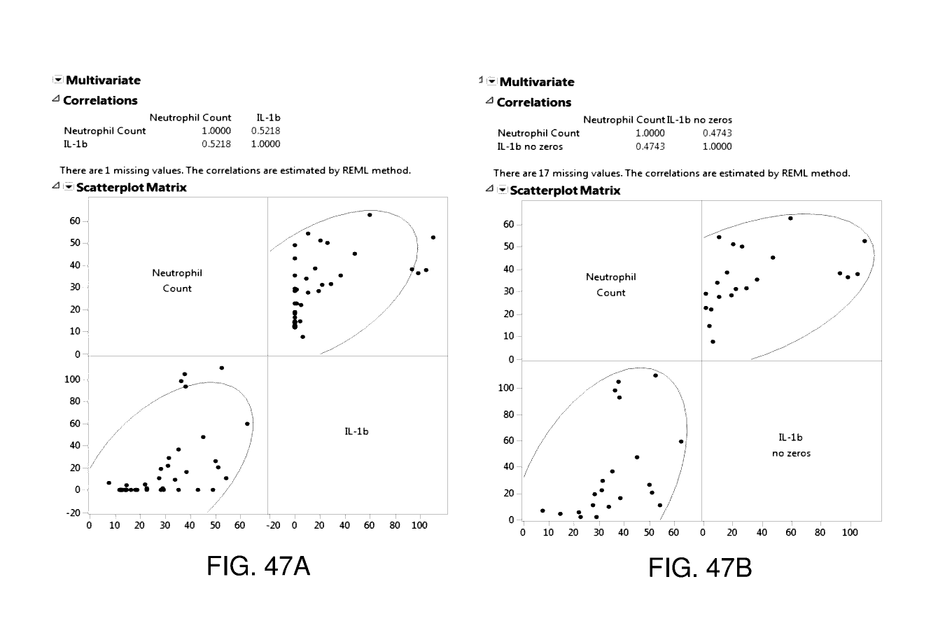

### DETAILED DESCRIPTION

In the following description, various aspects of the current subject matter are disclosed. For purposes of explanation, specific configurations and details are set forth in order to provide a thorough understanding of the current subject matter. However, it will also be apparent to one skilled in the art that the current subject matter can be practiced without the specific details presented herein. Furthermore, well known features can be omitted or simplified in order not to obscure the current subject matter.

### DEFINITIONS

All numerical designations (e.g., percentage, pH, dose, and concentration, including ranges) are approximations which are varied (+) or (−) by increments of 1.0 or 0.1, as appropriate. It is to be understood, although not always explicitly stated that all numerical designations are preceded by the term “about.” It also is to be understood, although not always explicitly stated, that the reagents described herein are merely exemplary and that equivalents of such are known in the art.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “subject” as used herein includes all members of the animal kingdom prone to suffering from the indicated disorder. In some aspects, the subject is a mammal, and in some aspects, the subject is a human of general population. The methods are also applicable to companion animals such as dogs and cats as well as livestock such as cows, horses, sheep, goats, pigs, and other domesticated and wild animals.

“Repair” or “healing” refers to restoration of some or all of a surface's cartilage to an acceptable operating condition. In some instances this may entail a 5%, 10%, 15%, 20%, 25%, 30%, 35%, 40%, 45%, 50%, 55%, 60%, 65%, 70%, 75%, 80%, 85%, 90%, 95%, 100%, and 100+% increase over the untreated condition in the patient in need of cartilage repair.

As used herein, “treatment” is an approach for obtaining beneficial or desired results, including clinical results. For purposes of this disclosure, beneficial or desired clinical results include, but are not limited to, alleviation or amelioration of symptoms (e.g., cartilage degeneration, soft tissue damage), diminishment of extent of inflammation, stabilized (i.e., not worsening) state of OA, delay or slowing of OA progression, amelioration or palliation of the OA state to include associated pain, and recovery (whether partial or total), whether detectable or undetectable.

As used here, “facilitating” or “promoting” refers to making the process of repair/healing easier in the presence of the bone marrow concentrate compared to the process of repair/healing without the bone marrow concentrate.

The term “therapeutically effective amount”, as used herein, refers to an amount of a pharmaceutical agent to treat or ameliorate an identified disease or condition (e.g., soft tissue damage, inflammation associated with surgery, skin regeneration, rheumatism, osteoarthritis and/or back symptoms), or to exhibit a detectable therapeutic or inhibitory effect, such as alleviation or amelioration of symptoms (e.g., soft tissue healing/repair, skin regeneration). The effect can be detected by any assay method known in the art. The precise effective amount for a subject will depend upon the subject's body weight, size, and health; the nature and extent of the condition; and the therapeutic selected for administration. Therapeutically effective amounts for a given situation can be determined by routine experimentation that is within the skill and judgment of the clinician.

“Whole cell platelet concentrate”, “platelet rich plasma”, and “platelet rich fibrin” are as generally known in the art. For example, platelet rich plasma or PRP can be obtained through methods as described in Landesberg et al., J Oral Maxillogac Surg 58:297, 2000, which is incorporated by reference in its entirety.

### Compositions and Methods of Production and Use

The invention described herein addresses the problem of rheumatism, osteoarthritis (e.g., soft tissue healing) and/or back symptoms and the failure to date of any effective therapeutics that can be prepared quickly and easily at a low cost.

Unlike known methods that require incubation or manipulation, the current subject matter's method can concentrate the IRAP from bone marrow for immediate use in a patient. The preparation method described herein is particularly advantageous, because it generate a bone marrow concentrate (also called bone marrow aspirate concentration or BMC or BMAC®) having a high level of IRAP, which means high level of therapeutic efficacy. Moreover, the preparation method described herein only takes 15 minutes to prepare a bone marrow concentrate for therapeutic uses, compared to 12-72 hours required by other methods (e.g., preparing autologous conditioned serum). Such quick preparation results implementation of therapeutic intervention within an hour, and makes it possible to carry out the treatment on site, such as on a field where athletes practice or compete (rather than performing the preparation in a lab far away). Furthermore, the bone marrow concentrate can be generated without the incubation of bone marrow with glass beads or similar activation devices, and therefore these products remain in compliance with associated regulatory restrictions.

Accordingly, provided herein is a method for preparing a bone marrow concentrate by (1) collecting a bone marrow sample from a subject, and (2) concentrating the bone marrow sample, thereby preparing the bone marrow concentrate, where the bone marrow concentrate comprises an autologous interleukin receptor antagonist protein (IRAP) concentrate comprising at least about 10,000 pg/mL of IRAP. The bone marrow concentrate is autologous.

In an illustrative embodiment, a bone marrow sample is collected as following: bone marrow is aspirated from the iliac crest into a 30 mL syringe containing at least one anticoagulant agent (e.g., acid citrate dextrose solution (ACD)). The needle is advanced 1 cm and rotated 90 degrees after each 5 mL aspirate until a total of 120 mL is aspirated in 4 syringes. The total aspirate is then mixed and concentrated accordingly to produce a bone marrow concentrate (“BMC”).

In some embodiments, spinning bone marrow sample in a centrifuge is used to create a bone marrow concentrate in the concentrating step of the method described herein. For example, the current subject matter can use the Arteriocyte Medical Systems, Inc. Magellan Autologous Platelet Separator, but broadly, the current subject matter relates to a method of centrifuging a bone marrow aspirate in any centrifuge to produce an autologous TRAP concentrate. Other exemplary commercial systems for generating a bone marrow concentrate include, but are not limited to, the Harvest SmartPReP 2 BMAC, Biomet BioCUE, Arthrex Angel, Celling Biosciences ART BMC, Emcyte PureBMC.

The centrifuge can allow for manual removal of the autologous RAP concentrate or it can be done through an automatic pathway where the concentrate is directly removed and put into a container such as a syringe for immediate use.

The centrifuge can include a sensor (light or laser) that allows for the detection of fluid density changes.

In some embodiments, the IRAP in a bone marrow concentrate produced according to the methods described herein has a concentration of at least about 12,000 pg/mL, about 13,000 pg/mL, about 14,000 pg/mL, about 15,000 pg/mL, about 16,000 pg/mL, about 17,000 pg/mL, about 18,000 pg/mL, about 19,000 pg/mL, about 20,000 pg/mL, about 25,000 pg/mL, about 30,000 pg/mL, about 35,000 pg/mL, about 40,000 pg/mL, about 45,000 pg/mL, about 50,000 pg/mL, or more.

In some embodiments, the bone marrow concentrate produced herein further includes at least one cytokine selected from the group consisting of PDGF, TGF-β1, TGF-β2, TGF-β3, IL-1β, and IL-8.

In some embodiments, the current subject matter can focus on the concentration of IRAP and/or serum containing cells secreting IRAP with centrifugation of autologous bone marrow aspirate. Bone marrow derived cells that include stem cells (mesenchymal, hematopoietic, and other progenitor cells) as well as platelets, leukocytes, and mono-nucleated cells. For example, the bone marrow concentrate produced herein may include stem cells (mesenchymal, hematopoietic, and other progenitor cells) as well as platelets, leukocytes, and/or mono-nucleated cells.

Also provided herein is a composition that includes a bone marrow concentrate produced according to the method described herein. The composition is autologous.

In some embodiments, the autologous composition includes stem cells (mesenchymal, hematopoietic, and other progenitor cells) as well as platelets, leukocytes, and/or mono-nucleated cells.

In some embodiments, the autologous composition includes an autologous TRAP concentrate produced according to the method described herein.

In some embodiments, the autologous composition includes TRAP having a concentration of at least about 12,000 pg/mL, about 13,000 pg/mL, about 14,000 pg/mL, about 15,000 pg/mL, about 16,000 pg/mL, about 17,000 pg/mL, about 18,000 pg/mL, about 19,000 pg/mL, about 20,000 pg/mL, about 25,000 pg/mL, about 30,000 pg/mL, about 35,000 pg/mL, about 40,000 pg/mL, about 45,000 pg/mL, about 50,000 pg/mL, or more.

In an exemplary embodiment, bone marrow aspirates and whole blood samples were analyzed using multiplex PCR analysis to evaluate levels of IRAP after commercially available methods for centrifugation were performed.

In some embodiments, the autologous IRAP concentrate can be combined with an agent to produce an autologous composition, where the agent is selected from the group consisting of: analgesic compounds, antibacterial compounds, antibiotics, antifungal compounds, anti-inflammatories, antiparasitic compounds, antiviral compounds, enzymes, enzyme inhibitors, glycoproteins, growth factors, hormones, steroids, glucocorticosteroids, immunomodulators, immunoglobulins, minerals, neuroleptics, proteins, lipoproteins, tumoricidal compounds, tumorstatic compounds, toxins and vitamins.

The current subject matter can be used broadly in any pathological scenario as a regenerative medicine biologic or biological adjunct. For way of illustration, but in no way limiting the examples, it can be used musculoskeletally, via injections, in surgical interventions where inflammation plays a role in pathology, intervention, treatment or recovery. It can also be used surgically for soft tissue repair for cartilage, tendon and muscle repair. It can be used for skin regeneration after surgical interventions.

For example, the current subject matter relates to a method for facilitating soft tissue healing in a subject in need thereof by administering to the subject a therapeutically effective amount of a composition (e.g., an autologous composition) comprising the bone marrow concentrate prepared according to the preparation method described above. For example, soft tissue includes cartilage, tendons, ligaments, fascia, skin, fibrous tissues, fat, and synovial membranes (which are connective tissue), and muscles, nerves and blood vessels (which are not connective tissue).

For example, the current subject matter relates to a method for treating osteoarthritis, rheumatism, osteoarthritis and/or back symptoms in a subject in need thereof by administering to the subject a therapeutically effective amount of a composition (e.g., an autologous composition) comprising the bone marrow concentrate prepared according to the preparation method described above.

For example, the current subject matter relates to a method for facilitating skin regeneration a subject in need thereof by administering to the subject a therapeutically effective amount of a composition (e.g., an autologous composition) comprising the bone marrow concentrate prepared according to the preparation method described above.

In some embodiments, the subject is suffering from soft tissue (e.g., cartilage, tendon and/or muscle) damage. In some embodiments, the subject is suffering from an inflammation associated with surgery. In some embodiments, the subject is suffering from or at risk of developing OA, rheumatism and/or back symptoms.

In some embodiments, the composition may also include an additional agent that is selected from the group consisting of: analgesic compounds, antibacterial compounds, antibiotics, antifungal compounds, anti-inflammatories, antiparasitic compounds, antiviral compounds, enzymes, enzyme inhibitors, glycoproteins, growth factors, hormones, steroids, glucocorticosteroids, immunomodulators, immunoglobulins, minerals, neuroleptics, proteins, lipoproteins, tumoricidal compounds, tumorstatic compounds, toxins and vitamins.

An autologous composition is formulated to be compatible with its intended route of administration. An autologous composition is preferably administered to a subject via intra-articular, or subcutaneous injection or infusion, or implant. Articulating joints, e.g., knee, elbow, shoulder, or hip are treated in this manner. Preferably, the autologous composition is administered locally, for example, directly to the diseased site (e.g., joint, skin) where repair or treatment is needed. Such methods are known in the art, e.g., as described in Wen et al., 2000, Am Fam Physician. 62:565-570 or Lockman et al., 2006, Can Fam Physician, 52: 1403-1404.

Other examples of routes of administration include parenteral, e.g., transdermal (i.e., topical) or local injection or infusion. Solutions or suspensions used for parenteral, intradermal, subcutaneous, local injection or infusion application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. For example a composition of the present invention is administered intraoperatively, arthroscopically, or by local direct injection into the affected tissue, such as a synovium of a joint.

Examples of dosing regimens that can be used in the methods of the invention include, but are not limited to, daily, three times weekly (intermittent), weekly, or every 14 days. In certain embodiments, dosing regimens include, but are not limited to, monthly dosing or dosing every 6 months. Typically, 1-3 injections are administered and about 2 weeks apart from each other (i.e., once about every two weeks and about 1-3 times in total). Typically, 1-2 injections are administered per year per articulating joint or until symptoms subside.

### Kits

Also provided herein are kits that include reagents/instruments for preparing a bone marrow concentrate and reagents/instrument for delivering (e.g., local injection) of the bone marrow concentrate. The kits may also include reagents/instrument for sterilization. Instructions (e.g., written, tape, VCR, CD-ROM, etc.) for carrying out the assay may be included in the kit.

The systems and methods disclosed herein can be embodied in various forms including, for example, a data processor, such as a computer that also includes a database, digital electronic circuitry, firmware, software, or in combinations of them. Moreover, the above-noted features and other aspects and principles of the present disclosed implementations can be implemented in various environments. Such environments and related applications can be specially constructed for performing the various processes and operations according to the disclosed implementations or they can include a general-purpose computer or computing platform selectively activated or reconfigured by code to provide the necessary functionality. The processes disclosed herein are not inherently related to any particular computer, network, architecture, environment, or other apparatus, and can be implemented by a suitable combination of hardware, software, and/or firmware. For example, various general-purpose machines can be used with programs written in accordance with teachings of the disclosed implementations, or it can be more convenient to construct a specialized apparatus or system to perform the required methods and techniques.

As used herein, the term “user” can refer to any entity including a person or a computer.

Although ordinal numbers such as first, second, and the like can, in some situations, relate to an order; as used in this document ordinal numbers do not necessarily imply an order. For example, ordinal numbers can be merely used to distinguish one item from another. For example, to distinguish a first event from a second event, but need not imply any chronological ordering or a fixed reference system (such that a first event in one paragraph of the description can be different from a first event in another paragraph of the description).

The foregoing description is intended to illustrate but not to limit the scope of the invention, which is defined by the scope of the appended claims. Other implementations are within the scope of the following claims.

To provide for interaction with a user, the subject matter described herein can be implemented on a computer having a display device, such as for example a cathode ray tube (CRT) or a liquid crystal display (LCD) monitor for displaying information to the user and a keyboard and a pointing device, such as for example a mouse or a trackball, by which the user can provide input to the computer. Other kinds of devices can be used to provide for interaction with a user as well. For example, feedback provided to the user can be any form of sensory feedback, such as for example visual feedback, auditory feedback, or tactile feedback; and input from the user can be received in any form, including, but not limited to, acoustic, speech, or tactile input.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations can be within the scope of the following claims.

### EXAMPLES

### Example 1

### Bone Marrow Concentrate and Platelet Rich Plasma Differ in Cell Distribution and Interleukin 1 Receptor Antagonist Protein Concentration

Biologic solutions to address the pathologic process of osteoarthritis (OA) have been investigated as potential treatments for focal cartilage lesions, osteochondral lesions, and generalized OA [1]. Mesenchymal stem cells (MSCs) appear promising for the treatment of OA, but are subject to regulatory restrictions, and are not currently approved by the FDA as well as many other countries' regulatory bodies for clinical use [6, 36]. Bone marrow concentrate (BMC) is generated by centrifugation of bone marrow aspirate (BMA) allowing for immediate administration to a patient, and many regulatory bodies have approved various companies' products for clinical use. In addition to MSCs, BMC contains numerous bioactive molecules and cell types including lymphocytes, neutrophils, monocytes, and platelets in various stages of differentiation [21, 23, 34]. In orthopedics, cytological analysis of BMC is rarely performed. In one study, a 4 fold increase in platelets, total nucleated cells, and CD34+ cells in BMC compared to BMA was reported [34]. The concentration of growth factors in BMC has not been quantified, despite their role in the regenerative potential of BMC [40, 47].

Platelet-rich plasma (PRP) is gaining popularity as a biologic treatment for focal cartilage defects and OA [13]. PRP is generated by centrifugation of peripheral blood resulting in increased platelet concentration. The platelets provide multiple growth factors with known roles in target cell activation, cell recruitment, cartilage matrix production, and modulation of the inflammatory response [22]. Currently the only established difference between PRP and BMC is the presence of MSCs in BMC, with no other information available on the component differences to guide clinicians. In current orthopedic practice, it is commonly regarded that BMC is essentially PRP with additional stem cells, however this has not been fully elucidated. Therefore, one aim of this study was to determine how BMC differs from PRP. There is known variability in PRP due to differences between patients and manufacturers, and the same premise is assumed for BMC, resulting in the second aim of this study, to compare BMCs generated from two different commercial systems.

### Materials and Methods

This study was prospective, approved by the hospital's Institutional Review Board, and supervised by the biologics committee as a quality assessment project. Between November 2013 and July 2014, 29 consecutive patients, within an age range of 18-70 years, consented for participation. All patients were under the care of a single surgeon. Patients were excluded if there was a history of blood disorders, hematological malignancy, use of immunosuppressive drugs, or drugs with bone marrow suppressive effects.

### Blood and Bone Marrow Aspirate Collection

Blood (25 mLs) was drawn into a syringe containing 4 mL acid citrate dextrose solution (ACD); the whole volume was mixed and 1 mL was removed for the study. Remaining blood was processed per manufacturer directions to generate 3 mLs PRP (Magellan®, Arteriocyte Medical Systems, Inc., Hopkinton, Mass.). One mL of PRP was removed for study purposes. Bone marrow was aspirated from the iliac crest into a 30 mL syringe containing 4 mL ACD. The needle was advanced 1 cm and rotated 90 degrees after each 5 mL aspirate until a total of 120 mL was aspirated in 4 syringes. The total aspirate was mixed, 1 mL was retained as the BMA sample for the study, and the remainder was separated into two 60 mL samples and processed in two systems; Magellan® (BMC-Y) (Arteriocyte Medical Systems Inc., Hopkinton, Mass.), and SmartPrep® 2 (BMC-X) (Harvest Technologies Corp., Plymouth, Mass.). All aspirations were performed by the same surgeon. The quantity of BMC produced varied from 3-7 mL as dictated by each separate system. One mL of each BMC-Y and BMC-X was retained for the study. Representatives from both BMC companies observed aspiration and concentration techniques to ensure compliance with company protocols. Samples were shipped de-identified with respect to patient and company to prevent any bias in sample processing and analysis. All samples were processed for analysis within 24 hours of collection.

### Cytology

Automated counting was performed on whole blood (WB), PRP, BMA, BMC-Y, and BMC-X samples to assess platelets, red blood cells (RBC), and nucleated cell counts (NCC), which included myeloid precursors as well as white blood cells (WBC): neutrophils, monocytes, lymphocytes, eosinophils, basophils. Cytological smears were evaluated in all samples to verify automated counts.

### Colony Forming Units and Flow Cytometry

Equal volumes of BMCs were used for CFU assays, rather than a known number of nucleated cells. This reflects clinical practice where volume, as opposed to cell count, would be the unit of BMC application. Samples of BMC were cultured in duplicate with DMEM containing 10% FBS, penicillin/streptomycin, and 1 ng/mL basic fibroblastic growth factor [50]. Media were changed every 48 hours. After two weeks, CFUs were circled with a 1.8 cm diameter self-inking marker. A colony was counted if it was ≧1 mm in diameter. This adapted technique [24, 33] allowed for retention of viable cells and further analyses.

Cells were then passaged once after colony counting at passage 0 and then lifted with Accumax (Sigma-Aldrich, St. Louis, Mo.) when confluent at passage 1 for flow cytometry of MSCs markers [16], including positive (CD146, CD73, CD271) and negative markers (CD34, CD45). Cells were pelleted in aliquots of 2.5×10⁵ to 1×10⁶ based on availability. Conjugated primary monoclonal antibodies and isotype controls were used as recommended by the manufacturer (BD Biosciences, San Jose, Calif.). Cells were analyzed on a FACS Canto II (BD Immunocytometry Systems, San Jose, Calif.) flow cytometer and using FlowJo software (TreeStar Inc., Ashland, Oreg.).

### Growth Factor and Cytokine Analyses

Aliquots of all samples were frozen for analysis of growth factors and cytokines important in cartilage repair and wound healing. Several, small aliquots were frozen to avoid free/thaw cycles which can adversely affect cytokine concentrations. Multiplex assays were performed on all samples according to manufacturer directions to measure vascular endothelial growth factor (VEGF), interleukin-1 beta (IL-1β), interleukin-6 (IL-6), interleukin-8 (IL-8), tumor necrosis factor-α (TNF-α), interleukin 1 receptor antagonist protein (IL-1ra), and interferon-γ (IFN-γ) (Fluorokine MAP Multiplex Human Cytokine Panel A, R&D Systems, Minneapolis, Minn.). Transforming growth factor-beta 1 (TGF-β1), TGF-β2, and TGF-β3 were measured using the Fluorokine MAP Mulitplex TGF-beta 3-plex Kit (R&D Systems, Minneapolis, Minn.). Platelet derived growth factor-BB (PDGF-BB), and acidic fibroblast growth factor (FGF-1) were quantified using the Fluorokine MAP Multiplex Human Angiogenesis Panel A (R&D Systems, Minneapolis, Minn.). Multiplex assays were performed using the Luminex 200 instrument (Luminex Corp., Austin, Tex.).

### Statistical Analyses

BMA was obtained from 29 patients and blood was also obtained from the last 14 of these patients. In BMC-X from the first 6 patients, no MSCs cells were observed in the cultures. The company was contacted, and they replaced the machine which resulted in growth of MSCs in all subsequent samples. The authors regarded this as mechanical failure and elected to exclude these patients and all their samples from the study to avoid introducing confounding variables and negatively skewing the data against the manufacturer of BMC-X. Four patients BMA and BMC samples were excluded because they were not processed within 24 hours of collection due to a shipping delay as the result of inclement weather. Further inclusion/exclusion criteria for statistical analysis were set to confirm that BMC and PRP were generated. For BMC, this was defined as an increase in NCC or CFU compared to BMA, resulting in exclusion of 1/19 of the remaining patients' samples. For PRP, inclusion was defined as an increase in platelet or PDGF concentration compared to that measured in whole blood, resulting in exclusion of 2/14 patients [13]. In order to account for the individual variability inherent in biologics, paired statistical methods were used to compare different biologics produced from the same individual. Without accounting for individual variability in colony forming units, cytology, and cytokines it would be difficult to determine the true differences in the biologics under study. The data differences between individual patient's samples in groups were tested for normality using a Shapiro-Wilk test. A two sided paired t-test was used for normally distributed differences between groups, otherwise, a Wilcoxon Signed Rank test was used, with an alpha of 0.05.

The mean age of patients was 48.2 years (range 23-68). Ten were female and the remainder (9) male. There were no reported complications associated with collection of bone marrow or blood.

### Results

### Cytology and Platelet Concentration in PRP Compared to BMC

BMC-Y was used for comparison to PRP because both were generated from the same system, thus allowing for a comparison based on biologic differences rather than preparation methods. The concentration of total WBCs and all subtypes of WBCs were greater in BMC-Y compared to PRP. There was an 11.8 fold increase in WBC concentration in BMC-Y compared to PRP. Neutrophils were increased in BMC-Y by 19 fold, monocytes by 11 fold, and lymphocytes by 7 fold compared to PRP. The platelet concentration in PRP was increased by 2.5 fold compared to WB, however, there was no difference in platelet concentration between BMC-Y and PRP.

### Cytology and Platelet Concentration in BMA Compared to BMC

The NCC in both BMC-Y and BMC-X was increased compared to BMA verifying that both systems concentrated nucleated cells. The NCC in BMC-Y was increased by 3.3 fold, and by 4.1 fold in BMC-X compared to BMA. There was no difference in NCC between BMC-Y and BMC-X. The total WBC concentration was increased in both BMC-Y and BMC-X compared to BMA, but there was no difference between BMC-Y and BMC-X. All subtypes of WBCs were increased in BMC-Y and BMC-X compared to BMA. The average neutrophil concentration in BMC-X was 1.6 fold greater than BMC-Y. In contrast, the average concentration of lymphocytes was 1.3 fold greater in BMC-Y compared to BMC-X. However, the paired sample analysis found there was no statistically significant difference between lymphocyte concentration in BMC-Y and BMC-X. There were no statistically significant differences in monocyte, eosinophil, or basophil concentrations between BMC-Y and BMC-X. Platelet concentration was increased by 4.8 fold in BMC-Y and 3.5 fold in BMC-X compared to BMA, resulting in a significantly greater platelet concentration in BMC-Y compared to BMC-X.

### Colony Forming Units

The number of CFUs was not different between duplicates (p=0.44), so duplicates were averaged for analyses. The mean number of CFUs was 7.8±standard deviation of 12.3 (range 0-46) in BMA, 41.4±27.4 (range 9-90) in BMC-Y, and 32.7±30.3 (range 2-90) in BMC-X. CFUs were significantly increased in both BMCs compared to BMA (p<0.0001), and BMC-Y and BMC-X were not significantly different (p=0.079).

Flow Cytometry of MSCs from BMC

The minimum cell number of 2.5×10⁵ needed for flow cytometry of each molecule limited the ability to measure all five MSC markers in every sample, so CD45 and CD271 were prioritized based on the literature [14, 45]. There were insufficient CFUs in BMA samples for comparison to BMC. All MSCs from BMC-Y and BMC-X were negative for the lymphocyte marker CD45. The majority of BMC-Y and BMC-X-derived MSCs were positive for CD271, and only a small percentage were positive for the hematopoietic stem cell marker CD34. All MSCs were positive for CD73 and variably positive for CD146.

### Growth Factors and Cytokines

### PDGF-BB

The concentration of PDGF was not significantly different between PRP and BMC-Y. As expected, PRP had increased (1.7 fold) PDGF compared to WB. Both BMC-Y and BMC-X had increased concentrations of PDGF compared to BMA, and BMC-Y had significantly more PDGF than BMC-X.

### TGF-β1, 2, 3

There was no significant difference in TGF-β1 concentration between BMC-Y and PRP. Not surprisingly, the concentration in PRP (average 72,788 pg/ml±40,582) was increased compared to WB (average 9,120 pg/ml±9,952). However, TGF-β1 was increased in both BMC-Y (average 112,793 pg/ml±63,165) and BMC-X (average 26,436 pg/ml±27,067) compared to BMA (average 5,006 pg/ml±3,403). BMC-Y had significantly more TGF-β1 compared to BMC-X. TGF-β2 was below limit of detection (17.5 pg/mL) in all BMA and WB samples. TGF-β2 was present in 15 out of 18 BMC-Y samples, 4 out of 19 BMC-X samples, and 9 out of 12 PRP samples. The concentration of TGF-β2 was greater in BMC-Y compared to PRP and BMC-X. TGF-β3 concentrations were below the limit of detection (62.15 pg/mL) in all samples.

### VEGF

VEGF concentrations were lowest in PRP, WB, and BMA samples. In BMC-Y, VEGF was increased compared to PRP. Compared to BMA, VEGF concentration was increased 4 fold in BMC-Y and 7 fold in BMC-X.

### IL-8

IL-8 concentrations were below the limit of detection (3.5 pg/mL) in WB and PRP samples. IL-8 was increased 3 fold in BMC-Y and 5 fold in BMC-X samples compared to BMA. There was no difference in IL-8 concentration between the two BMC groups.

### IL-1β

There was no measurable IL-1β in WB, PRP, or BMA. Additionally, IL-1β was undetectable in nine BMC-Y and six BMC-X samples. In the samples that did have a concentration greater than the lower limit of detection (1.5 pg/mL), IL-1β was greater in BMC-X compared to BMC-Y.

### IL-1ra

All samples contained measurable IL-1ra. IL-1ra was significantly increased in BMC-Y (average 13,432 pg/ml±8,588) compared to PRP (average 588 pg/ml±457). In WB (2,388 average pg/ml±1,744), IL-1ra was greater than in PRP. Compared to BMA (average 4,510 pg/ml±2,994), IL-1ra was significantly increased in both BMC-Y (3 fold) and BMC-X (5 fold), and BMC-X (average 21,179 pg/ml±9,563) was significantly greater than BMC-Y. The ratios of IL-1ra to IL-1β were calculated for all samples with detectable concentrations of IL-1β. The average ratio of IL-1ra:IL-1β for the 6 patients with BMC-Y and BMC-B samples with detectable IL-1β was not significantly different (p=0.09).

### TNF-α, IL-6, IFN-γ, FGF-1

All four of these cytokines were undetectable in all samples. Minimum detectable concentrations based on the standard curves were: TNF-α (5.14 pg/mL), IL-6 (5.16 pg/mL), IFN-γ (2.85 pg/mL), and FGF-1 (5.66 pg/mL).

### Discussion

The demand and use of biological adjuncts in orthopedics is growing rapidly [20, 21]. Despite this growth, however, there is a paucity of data detailing the true composition and differences in the biologics currently available. This is the first study to characterize and compare PRP and BMC from the same patient cohort and the first report to document IL-1ra in BMA and BMC. The data presented herein suggest there are several significant differences in the cellular and molecular composition of PRP and BMC. Additionally, there were differences in cytology and bioactive molecules between BMC manufacturing systems.

Both BMC manufacturing systems were capable of effectively concentrating BMA as demonstrated by an increased total NCC in most BMC samples.

Although there was an increase in NCC in the BMC-X group compared to BMC-Y, BMC-Y and BMC-X did not have significantly different CFUs. This suggests that NCC alone may not be predictive of MSC concentration. In a previous study, higher NCCs and CFUs were found in BMC-X compared to BMC-Y [33]. These conflicting results are likely explained by different study methodologies. In the current study, a uniform volume of BMC was used for culture rather than a defined number of nucleated cells [33]. This method was chosen to reflect clinical practice where a doctor would administer a specified volume of BMC rather than a defined number of nucleated cells. All cultured cells had flow cytometry markers consistent with MSCs. The heterogeneity in surface markers of MSCs observed in this study has been previously reported and is not surprising given the lack of cell selection processes and the short culture duration (analysis at passage 1) as some of the characteristic markers are not uniformly expressed until later passages [3, 38].

Several growth factors have positive effects on cartilage repair and in the treatment of OA. The beneficial role of PDGF in cartilage repair and homeostasis has been extrapolated based on its function during chondrogenesis in early limb development [2]. PDGF induces MSC proliferation [22, 25] and inhibits IL-1β induced chondrocyte apoptosis and inflammation [42]. PDGF was present in all samples in varying concentrations; centrifugation of either BMA or WB resulted in an increase in PDGF concentrations, corresponding to increased platelet concentrations. All three TGF-β isoforms examined in this study have roles in chondrogenesis [7, 35, 39, 43, 44, 51, 56]. TGF-β1 has been shown to stimulate chondrogenesis of synovium and bone marrow-derived MSCs [18, 37], inhibit IL-1β-mediated inflammation [9], and enhance cartilage healing [12]. TGF-β1 was increased in PRP and BMC, paralleling increased platelet concentrations. PRP and BMC-Y did not differ in TGF-β1 concentration, however increased TGF-β1 was present in BMC-Y compared to BMC-X. These findings are related to platelet, monocyte, and neutrophil concentrations [31]. TGF-β2 was present in most BMC-Y and PRP samples, but only a few BMC-X samples. In OA chondrocytes, TGF-β2 decreases collagen type 2 cleavage and chondrocyte hypertrophy through inhibition of IL-1β and TNF-α [49]. Many cell types secrete TGF-β2, thus differences in cellular distribution may be the source of the divergent TGF-β2 concentrations.

In balance with growth factors, PRP and BMC products contain pro-inflammatory cytokines. IL-1β and IL-8 are secreted by monocytes, neutrophils, and MSCs [19, 29, 52]. In WB and PRP, IL-1β and IL-8 were undetectable, but they were present in BMA and increased in both BMC groups. This is a result of concentration of neutrophils, monocytes, and MSCs during centrifugation [4]. IL-8 is a potent chemoattractant for neutrophils, which secrete IL-1, and in turn, can further stimulate monocytes to produce IL-8 [4, 8, 32]. This shows that PRP is a less inflammatory, more anabolic biologic. However, the surprising and significant concentration of IL-1ra found in BMC and its absence in PRP needs to be taken into consideration when choosing between PRP and BMC for any therapeutic modality.

Interleukin-1 receptor antagonist (IL-1ra) is a naturally occurring antagonist that competitively binds to IL-1β and IL-1a cell surface receptors thereby inhibiting IL-1 catabolism. IL-1ra is thought to be responsible for the beneficial effects of the biologic autologous conditioned serum (ACS) [54], which is produced by incubation of blood with glass beads [41]. Two randomized control trials in knee OA reported superiority of ACS over hyaluronan or saline injection [5, 55] in patient reported standardized outcome scores. This has resulted in widespread use of the commercial ACS product “Orthokine®” (autologous conditioned serum, Germany) as an effective method for treating OA of the knee. ACS is not approved for use in the United States, thus its implementation has been confined primarily to Europe. The original reported mean concentration of IL-1ra in ACS was 10,254 pg/mL [41], and subsequent studies documented concentrations of 2,015 pg/mL [54] and <2,000 pg/mL [46]. In the present study, the average concentration of IL-1ra in BMA was 4,510 pg/mL, which was then increased 3 fold in BMC-Y and 5 fold in BMC-X. Such high concentration of IL-1ra made BMC a great therapeutic composition for treating soft tissue healing, such as OA. Since these BMC products can be generated without the incubation of blood with glass beads, these products remain in compliance with associated regulatory restrictions. For effective therapy, an IL-1ra:IL-1 ratio of 10:1 to 100:1[15, 30] has been reported as sufficient to block IL-1. The IL-1ra:IL-1β ratio in BMC samples ranged from 249:1 to 17,568:1, indicating a net inhibitory effect on IL-1. PRP and WB had lower concentrations of IL-1ra compared to BMA and BMC products. This may be attributed to lower concentrations of nucleated cells capable of producing IL-1ra, or to the different status of nucleated cells in the bone marrow compared to in peripheral blood.

The differences in cellular and molecular components described in these biologics highlight the complexity of these modalities.

### CONCLUSION

There are significant differences in the cellular and molecular composition of autologous biologic products. Thus, it is important that their differences are considered in light of the pathology and patient it is being used for. More importantly, BMC provides a readily available source to deliver the potent anti-inflammatory therapy, IL-1ra, to patients where this product is not otherwise available due to regulatory restrictions.

### Ethical Standards:

All procedures performed in studies involving human participants were approved by the institutional research review board and in accordance with the ethical standards of the 1964 Helsinki declaration and its later amendments or comparable ethical standards.

Informed consent was obtained from all individual participants included in the study.

TABLE 1

| | | | | | | | Nucleated | | |
|---|---|---|---|---|---|---|---|---|---|
| | Platelet Count | | | | | | Cell Count | | |
| Patient | Whole | PRP | Fold Change | BMA | X | Y | BMA | X | Y |
| 16 | 30 | 280 | 9.3 | 57 | 105 | 74 | 10.8 | 46.9 | 25.1 |
| 17 | 98 | 58 | 0.6 | 12 | 27 | 47 | 6.2 | 15.2 | 24 |
| 18 | 178 | 193 | 1.1 | 94 | 300 | 304 | 12 | 49.5 | 19.8 |
| 19 | 14 | 112 | 8.0 | 15 | 11 | 127 | 6.1 | 22.8 | 44.9 |
| 20 | 37 | 261 | 7.1 | 40 | 218 | 226 | 12 | 46.7 | 64.7 |
| 21 | 32 | 73 | 2.3 | 28 | 23 | 53 | 10.7 | 32.1 | 18.2 |
| 22 | 41 | 216 | 5.3 | 45 | 180 | 213 | 12.2 | 50.2 | 42.7 |
| 23 | 130 | 163 | 1.3 | 65 | 276 | 362 | 23.2 | 81.4 | 94 |
| 24 | 80 | 122 | 1.5 | 65 | 158 | 229 | 902 | 43.2 | 46.6 |
| 25 | ND | 243 | | 56 | 249 | 271 | 5.9 | 31.5 | 27.2 |
| 26 | 94 | 128 | 1.4 | 26 | 30 | 118 | 6.7 | 20.2 | 18.6 |
| 27 | 59 | 264 | 4.5 | 19 | 47 | 14 | 21.5 | 67.2 | 10.5 |
| 28 | 171 | 368 | 2.2 | 5 | 55 | 80 | 16.7 | 67.1 | 38.6 |
| 29 | ND | 248 | | 14 | 26 | 330 | 13.9 | 62.3 | 61.2 |

| | | |
|---|---|---|
| Clumping | Count is lower than | *Need to ask |
| | indicated based on | about 902 |
| | smear estimation | |
| | Count is higher | |
| | than indicated | |
| | based on smear | |
| | estimation | |
| | Clumping | |

TABLE 2
Jennifer Cassano/Dr. Fortier
Hu Angiogenesis Base Kit A (R&D)
Jun. 24, 2014
19 BMA C-X the well leaked during the read so this resulted in poor bead
recovery & no result to reportNot able to repeat due to low reagent volumes.

| | FGF acidic (33) | | | PDGF-BB (37) | | |
|---|---|---|---|---|---|---|
| Description | Exp Conc | Obs Conc | FI | Exp Conc | Obs Conc | FI |
| Blank | | | 3 | | | 2.6 |
| Standard 1 | 23800 | 23793.08 | 8753 | 21100 | 20569.14 | 18907 |
| Standard 2 | 5950 | 5955.06 | 4565 | 5275 | 5355.18 | 15002 |
| Standard 3 | 1487.5 | 1483.26 | 1564.5 | 1318.75 | 1300.44 | 7350.5 |
| Standard 4 | 371.88 | 374.99 | 466 | 329.69 | 335.53 | 2745 |
| Standard 5 | 92.97 | 91.47 | 145.5 | 82.42 | 81.51 | 942 |
| Standard 6 | 23.24 | 23.85 | 64 | 20.61 | 20.63 | 363.5 |
| Standard 7 | 5.81 | 5.66 | 41 | 5.15 | 5.16 | 171 |

| | |
|---|---|
| FGF acidic (33) | PDGF-BB (37) |

| Description | Obs Conc | FI | Obs Conc | FI |
|---|---|---|---|---|
| 8 BMA | OOR < | 6 | 2637.52 | 3863.5 |
| 9 | OOR < | 6 | 3435.29 | 4698.5 |
| 10 | OOR < | 7 | 640.3 | 1319.5 |
| 14 | OOR < | 4.5 | 379.6 | 894 |
| 15 | OOR < | 4 | 811.84 | 1579 |
| 16 | OOR < | 5 | 2233.54 | 3410 |
| 17 | OOR < | 5 | 2115.04 | 3272.5 |
| 18 | OOR < | 4.5 | 3819.37 | 5075.5 |
| 19 | OOR < | 6.5 | 864.37 | 1656 |
| 20 | OOR < | 6 | 1990.82 | 3126 |
| 21 | OOR < | 5 | 1791.87 | 2886 |
| 22 | OOR < | 5.5 | 2527.75 | 3742.5 |
| 23 | OOR < | 7 | 2271.85 | 3454 |
| 24 | OOR < | 4 | 583.53 | 1230.5 |
| 25 | OOR < | 3 | 2023.69 | 3165 |
| 26 | OOR < | 6 | 1967.74 | 3098.5 |
| 27 | OOR < | 5 | 2528.65 | 3743.5 |
| 28 | OOR < | 8 | 919.35 | 1735.5 |
| 29 | OOR < | 4 | 1049.76 | 1920 |
| 8 BMA C-X | OOR < | 5 | 3486.86 | 4750 |
| 9 | OOR < | 5 | 7358.52 | 7967.5 |
| 10 | OOR < | 8 | 4790.31 | 5966 |
| 14 | OOR < | 3 | 5068.96 | 6206.5 |
| 15 | OOR < | 3 | 2295.45 | 3481 |
| 16 | OOR < | 6 | 6269.93 | 7175 |
| 17 | OOR < | 2 | 1525.26 | 2553 |
| 18 | OOR < | 4 | 16029.84 | 12295.5 |
| 19 | *** | *** | *** | *** |
| 20 | OOR < | 6.5 | 7822.16 | 8283 |
| 21 | OOR < | 4 | 618.16 | 1285 |
| 22 | OOR < | 5 | 2602.44 | 3825 |
| 23 | OOR < | 11 | 8534.94 | 8744.5 |
| 24 | OOR < | 3 | 2678.26 | 3908 |
| 25 | OOR < | 5 | 8057.58 | 8438.5 |
| 26 | OOR < | 7 | 3251.38 | 4512.5 |
| 27 | OOR < | 12.5 | 5189.38 | 6308.5 |
| 28 | OOR < | 7 | 2435.01 | 3639 |
| 29 | OOR < | 4.5 | 2642.55 | 3869 |
| 8 BMA C-Y | OOR < | 9 | 9611.24 | 9391.5 |
| 9 | OOR < | 6 | 4737.84 | 5920 |
| 10 | OOR < | 11 | 7740.74 | 8228.5 |
| 14 | OOR < | 5 | 11113.28 | 10205 |
| 15 | OOR < | 4 | 4460.54 | 5673 |
| 16 | OOR < | 8 | 6404.34 | 7277 |
| 17 | OOR < | 6 | 6522.27 | 7365.5 |
| 18 | OOR < | 4 | 15055.52 | 11939.5 |
| 19 | OOR < | 10 | 9535.82 | 9348 |
| 20 | OOR < | 5 | 10114.41 | 9675 |
| 21 | OOR < | 6 | 2433.22 | 3637 |
| 22 | OOR < | 7 | 7040.91 | 7744 |
| 23 | OOR < | 11 | 10509.15 | 9889.5 |
| 24 | OOR < | 3 | 3896.58 | 5149.5 |
| 25 | OOR < | 3 | 12912.4 | 11061 |
| 26 | OOR < | 10 | 2910.32 | 4157.5 |
| 27 | OOR < | 3.5 | 9438.55 | 9291.5 |
| 28 | OOR < | 6 | 3305.88 | 4568 |
| 29 | OOR < | 5 | 7568.59 | 8112 |
| 16 PRP | OOR < | 3 | 6991.26 | 7708.5 |
| 17 | OOR < | 2.5 | 600.31 | 1257 |
| 18 | OOR < | 3 | 4060.82 | 5305 |
| 19 | OOR < | 3 | 3597.44 | 4859.5 |
| 20 | OOR < | 4 | 7277.4 | 7911 |
| 21 | OOR < | 4 | 5216.13 | 6331 |
| 22 | OOR < | 3 | 8416.16 | 8669.5 |
| 23 | OOR < | 3.5 | 5225.65 | 6339 |
| 24 | OOR < | 3 | 3837.06 | 5092.5 |
| 25 | OOR < | 3 | 3747.41 | 5006 |
| 26 | OOR < | 3 | 2277.09 | 3460 |
| 27 | OOR < | 13 | 4327.37 | 5552 |
| 28 | OOR < | 4 | 10679.5 | 9980 |
| 29 | OOR < | 3 | 9804.05 | 9501.5 |
| 16 Whole Blood | OOR < | 3 | 2397.67 | 3597 |
| 17 | OOR < | 3 | 2063.49 | 3212 |
| 18 | OOR < | 3 | 5212.56 | 6328 |
| 19 | OOR < | 4 | 842.45 | 1624 |
| 20 | OOR < | 3 | 3983.38 | 5232 |
| 21 | OOR < | 4 | 3111 | 4368 |
| 22 | OOR < | 3 | 2572.48 | 3792 |
| 23 | OOR < | 4 | 5025.03 | 6169 |
| 24 | OOR < | 3 | 6179.26 | 7105.5 |
| 25 | OOR < | 4 | 7697.65 | 8199.5 |
| 26 | OOR < | 4 | 994.16 | 1842 |
| 27 | OOR < | 3 | 1247.42 | 2190 |
| 28 | OOR < | 3 | 5179.89 | 6300.5 |
| 29 | OOR < | 3 | 2915.03 | 4162.5 |

TABLE 3

| patient | prp | | | whole blood | | | bmaC-γ | | | BMACX | | | bma | | |
|---|---|---|---|---|---|---|---|---|---|---|---|---|---|---|---|
| # | wbc | rbc | neutrophils | wbc | rbc | neutrophils | wbc | rbc | neutrophils | wbc | rbc | neutrophils | wbc | rbc | neutrophils |
| 16 | 3.7 | 1.8 | 1.1 | 3.3 | 4.3 | 1.7 | | 1120 | 14.307 | | 3689 | 35.175 | | 4053.1 | 7.668 |
| 17 | 1.2 | 1.6 | 0.6 | 2.1 | 2.3 | 1.2 | | 171 | 14.832 | | 2786 | 12.16 | | 4459.1 | 4.6995 |
| 18 | 2.1 | 0.8 | 1.1 | 6.2 | 3.1 | 4.5 | | 494 | 11.979 | | 3650 | 36.3825 | | 3425.4 | 9.78 |
| 19 | 3.9 | 1 | 0.9 | 2.5 | 4.6 | 1.3 | | 1051 | 29.185 | | 2455 | 13.8624 | | 3236.1 | 4.331 |
| 20 | 2.9 | 0.8 | 0.8 | 6.2 | 4.4 | 4.2 | | 206 | 45.29 | | 2858 | 38.5275 | | 3748.1 | 9.66 |
| 21 | 2.4 | 0.8 | 0.8 | 3.8 | 4.6 | 2.5 | | 737 | 12.922 | | 4800 | 22.6305 | | 4124.7 | 7.0941 |
| 22 | 4.3 | 0.9 | 0.8 | 6.4 | 4 | 3.6 | | 843 | 31.598 | | 3320 | 3.514 | | 3371.6 | 8.54 |
| 23 | 2.2 | 0.9 | 0.8 | 4.2 | 3 | 2.6 | | 638 | 53.58 | | 1825 | 62.5152 | | 3349.3 | 17.1216 |
| 24 | 3.3 | 0.8 | 0.6 | 4.3 | 3.8 | 1.9 | | 551 | 28.193 | | 2117 | 29.376 | | 3957.6 | 631.4 |
| 25 | 3.8 | 0.8 | 0.5 | 2.9 | 4.4 | 1.8 | | 737 | 16.4016 | | 2657 | 21.684 | | 4500 | 4.2775 |
| 26 | 0.7 | 0.4 | 0.1 | 1.9 | 2 | 0.7 | | 651 | 12.548 | | 2650 | 14.5844 | | 3583.3 | 4.6755 |
| 27 | 82.8 | 0.5 | 59.7 | 5.6 | 5 | 4 | | 950 | 7.5915 | | 2983 | 50.064 | | 4308.5 | 16.942 |
| 28 | 4 | 0.9 | 1.8 | 6 | 4 | 4.5 | | 3189 | 22.8898 | | 2534 | 48.8488 | | 3563.9 | 11.2391 |
| 29 | 5.1 | 0.9 | 1.2 | 4 | 4.2 | 2.5 | | 564 | 85.496 | | 2901 | 43.1116 | | 3622.2 | 10.7725 |
| | | | 5.0571429 | | | 2.6071429 | | | | | | | | | |

TABLE 4
Jennifer Cassano/Dr. Fortier -
Repeat
Hu Cytokine Base Kit (R&D)
Jun. 18, 2014
Repeat of highlighted samples in “HuCyto Base”

| | IL-1B/IL-1F2 (6) | | | IL-1ra/IL-1F3 (16) | | | IL-6 (32) | | | CXCL8/IL-8 (36) | | |
|---|---|---|---|---|---|---|---|---|---|---|---|---|
| | Exp | Obs | | Exp | Obs | | Exp | Obs | | Exp | Obs | |
| Description | Conc | Conc | FI | Conc | Conc | FI | Conc | Conc | FI | Conc | Conc | FI |
| Blank | | | 2 | | | 7 | | | 1 | | | 2 |
| Standard 1 | 2050 | 2202.61 | 2553 | 1750 | 1905.04 | 2399 | 4050 | 4264.86 | 5545 | 2850 | 2973.43 | 8295 |
| Standard 2 | 653.33 | 608.72 | 705 | 583.33 | 507.83 | 759.5 | 1350 | 1243.51 | 1812 | 950 | 906.93 | 3659 |
| Standard 3 | 227.78 | 245 | 293 | 194.44 | 216.87 | 371.5 | 450 | 459.11 | 733 | 316.7 | 290.78 | 1459.5 |
| Standard 4 | 75.93 | 71.48 | 91 | 64.81 | 57.75 | 134 | 150 | 156.25 | 282 | 105.6 | 163.91 | 587 |
| Standard 5 | 25.31 | 25.76 | 39 | 21.6 | 27.04 | 82 | 50 | 46.22 | 105.5 | 35.19 | 24.84 | 219 |
| Standard 6 | 8.44 | 11.63 | 23 | 7.2 | 8.36 | 47 | 16.67 | 20.75 | 62 | 11.73 | 19.22 | 195.5 |
| Standard 7 | 2.81 | 1.85 | 12 | 2.4 | 1.46 | 32 | 5.56 | 4.65 | 33 | 3.91 | 2.14 | 141 |

| | TNF-a (77) | | | VEGF (52) | | | IFN-γ (75) | | |
|---|---|---|---|---|---|---|---|---|---|
| Description | Exp Conc | Obs Conc | FI | Exp Conc | Obs Conc | FI | Exp Conc | Obs Conc | FI |
| Blank | | | 3 | | | 7 | | | 4 |
| Standard 1 | 3950 | 3974.11 | 7051 | 2500 | 2610.69 | 6794 | 2200 | 2245.56 | 7540 |
| Standard 2 | 1316.67 | 1287.62 | 2569 | 833.33 | 899.51 | 2109.5 | 733.33 | 681.71 | 3333.5 |
| Standard 3 | 438.89 | 443.93 | 919 | 277.78 | 207.59 | 468.5 | 244.44 | 276.12 | 1716.5 |
| Standard 4 | 145.3 | 162.52 | 342 | 92.59 | 223.77 | 503 | 81.48 | 78.33 | 649 |
| Standard 5 | 48.77 | 39.69 | 96.5 | 30.85 | 36.72 | 132 | 27.16 | 25.99 | 274.5 |
| Standard 6 | 16.26 | 26.16 | 71 | 10.29 | 37.01 | 132.5 | 9.05 | 9.67 | 134 |
| Standard 7 | 5.42 | 3.93 | 32 | 3.43 | OOR < | 63 | 3.02 | 2.93 | 65 |

| | IL-1B/ | | | | | | | | | | | | | |
|---|---|---|---|---|---|---|---|---|---|---|---|---|---|---|
| | IL-1F2 | | IL-1ra/IL-1F3 | | IL-6 | | CXCL8/ | | TNF-a | | VEGF | | | |
| | (6) | | (16) | | (32) | | IL-8 (36) | | (77) | | (52) | | IFN-γ (75) | |
| | Obs | | Obs | | Obs | | Obs | | Obs | | Obs | | Obs | |
| Description | Conc | FI | Conc | FI | Conc | FI | Conc | FI | Conc | FI | Conc | FI | Conc | FI |
| 28 BMA | OOR < | 6 | *2703.20 | 3259 | OOR < | 1 | 65.33 | 409 | OOR < | 4 | 126.71 | 501.5 | *** | *** |
| 22 BMA | 10.74 | 22 | *5048.53 | 5656 | OOR < | 2 | 101.85 | 588 | OOR < | 3 | 205.3 | 470 | OOR < | 0 |
| C-Y | | | | | | | | | | | | | | |
| 17 Whole | OOR < | 0 | 100.55 | 201.5 | OOR < | 1 | OOR < | 48 | OOR < | 2 | OOR < | 21 | OOR < | 5 |
| Blood | | | | | | | | | | | | | | |

TABLE 5

| | Count | | | | Total | | | | | |
|---|---|---|---|---|---|---|---|---|---|---|
| | Date | Type | Plate | Cell Count | Colonies | Average | Small | Med | Large | |
| 8 | Feb. 25, 2014 | BMA | 1 | too few to | 1 | | | 1 | | |
| | | | | count | | | | | | |
| | | BMA | 2 | too few to | 1 | 1 | | 1 | | |
| | | | | count | | | | | | |
| | | X | 1 | 22500 | 8 | | 3 | 2 | 3 | |
| | | X | 2 | 25000 | 7 | 7.5 | 4 | 1 | 2 | |
| | | Y | 1 | too much | 23 | | 2 | 3 | 18 | |
| | | | | debri to count | | | | | | |
| | 400 | Y | 2 | 120000 | 30 | 26.5 | | 3 | 27 | |
| 9 | Feb. 25, 2014 | BMA | 1 | too few to | 2 | | 1 | 1 | | |
| | | | | count | | | | | | |
| | | BMA | 2 | too few to | 5 | 3.5 | | 3 | 2 | |
| | | | | count | | | | | | |
| | | X | 1 | 400000 | 25 | | 4 | 4 | 17 | |
| | | X | 2 | 30000 | 26 | 25.5 | 2 | 6 | 18 | |
| | | Y | 1 | 60000 | 19 | | 4 | 7 | 8 | |
| | | Y | 2 | 120000 | 10 | 14.5 | 0 | 3 | 7 | |
| 10 | Mar. 11, 2014 | BMA | 1 | 65000 | 11 | | 5 | 3 | 3 | |
| | | BMA | 2 | 85000 | 16 | 13.5 | 8 | 7 | 1 | |
| | | X | 1 | 10000000 | 46 | | | | 46 | |
| | | X | 2 | 6500000 | 46 | 46 | | | 46 | |
| | | Y | 1 | 9200000 | 36 | | | | 36 | |
| | | Y | 2 | 9000000 | 36 | 36 | | | 36 | |
| 14 | Mar. 25, 2014 | BMA | 1 | too few to | 1 | | 0 | 1 | 0 | |
| | | | | count | | | | | | |
| | | BMA | 2 | too few to | 1 | 1 | 0 | 1 | 0 | |
| | | | | count | | | | | | |
| | | X | 1 | 260000 | 21 | | 5 | 7 | 9 | |
| | | X | 2 | 260000 | 36 | 28.5 | 0 | 9 | 27 | |
| | | Y | 1 | 400000 | 24 | | 0 | 8 | 16 | |
| | | Y | 2 | 400000 | 23 | 23.5 | 1 | 5 | 17 | |
| 15 | Mar. 25, 2014 | BMA | 1 | too few to | 1 | | 0 | 0 | 1 | |
| | | | | count | | | | | | |
| | | BMA | 2 | too few to | 1 | 1 | 0 | 1 | 0 | |
| | | | | count | | | | | | |
| | | X | 1 | 150000 | 25 | | 2 | 6 | 17 | |
| | | X | 2 | 150000 | 24 | 24.5 | 2 | 7 | 15 | |
| | | Y | 1 | 200000 | 41 | | 5 | 22 | 14 | |
| | | Y | 2 | 200000 | 42 | 41.5 | 10 | 15 | 17 | |
| 16 | Apr. 3, 2014 | BMA | 1 | none seen | 0 | | 0 | 0 | 0 | |
| | | BMA | 2 | too few to | 1 | 0.5 | 0 | 1 | 0 | |
| | | | | count | | | | | | |
| | | X | 1 | 65000 | 24 | | 6 | 5 | 13 | |
| | | X | 2 | 65000 | 29 | 26.5 | 14 | 4 | 11 | |
| | | Y | 1 | 95000 | 44 | | 13 | 15 | 16 | |
| | | Y | 2 | 95000 | 28 | 36 | 11 | 13 | 4 | |
| 17 | Apr. 3, 2014 | BMA | 1 | too few to | 1 | | 1 | 0 | 0 | |
| | | | | count | | | | | | |
| | | BMA | 2 | too few to | 1 | 1 | 0 | 1 | 0 | |
| | | | | count | | | | | | |
| | | X | 1 | none seen | 0 | | 0 | 0 | 0 | |
| | | X | 2 | too few to | 3 | 1.5 | 0 | 2 | 1 | |
| | | | | count | | | | | | |
| | | Y | 1 | 31000 | 9 | | 1 | 6 | 2 | |
| | | Y | 2 | 31000 | 9 | 9 | 4 | 5 | | |
| 18 | Apr. 16, 2014 | BMA | 1 | 60000 | 7 | | 0 | 3 | 4 | |
| | | BMA | 2 | 60000 | 10 | 8.5 | 4 | 3 | 3 | |
| | | X | 1 | 1300000 | 73 | | 1 | 7 | 65 | |
| | | X | 2 | 700000 | also | 73 | | | | |
| | | | | | confluent | | | | | |
| | | Y | 1 | 800000 | 64 | | 1 | 8 | 55 | |
| | | Y | 2 | 1200000 | also | 64 | | | | |
| | | | | | confluent | | | | | |
| 19 | Apr. 17, 2014 | BMA | 1 | none seen | 0 | | | | | |
| | | BMA | 2 | none seen | 0 | 0 | | | | |
| | | X | 1 | too few to | 6 | | 3 | 2 | 1 | |
| | | | | count | | | | | | |
| | | X | 2 | too few to | 5 | 5.5 | 1 | 3 | 1 | |
| | | | | count | | | | | | |
| | | Y | 1 | 46000 | 30 | | 10 | 13 | 7 | |
| | | Y | 2 | 38000 | 24 | 27 | 7 | 12 | 5 | |
| 20 | Apr. 17, 2014 | BMA | 1 | too few to | 4 | | 2 | 1 | 1 | |
| | | | | count | | | | | | |
| | | BMA | 2 | too few to | 7 | 5.5 | 4 | 2 | 1 | |
| | | | | count | | | | | | |
| | | X | 1 | 10000 | 6 | | 4 | 0 | 2 | |
| | | X | 2 | 10000 | 16 | 11 | 8 | 4 | 4 | |
| | | Y | 1 | 180000 | 51 | | 10 | 22 | 19 | |
| | | Y | 2 | 220000 | 63 | 57 | 19 | 16 | 28 | |
| 21 | Apr. 29, 2014 | BMA | 1 | too few to | 7 | | 2 | 0 | 5 | |
| | | | | count | | | | | | |
| | | BMA | 2 | too few to | 5 | 6 | 0 | 1 | 4 | |
| | | | | count | | | | | | |
| | | X | 1 | too few to | 7 | | 3 | 1 | 3 | |
| | | | | count | | | | | | |
| | | X | 2 | too few to | 5 | 6 | 1 | 3 | 1 | |
| | | | | count | | | | | | |
| | | Y | 1 | 130000 | 20 | | 5 | 11 | 4 | |
| | | Y | 2 | 120000 | 18 | 19 | 8 | 2 | 8 | |
| 22 | Apr. 29, 2014 | BMA | 1 | too few to | 2 | | 2 | 0 | 0 | |
| | | | | count | | | | | | |
| | | BMA | 2 | too few to | 3 | 2.5 | 1 | 0 | 2 | |
| | | | | count | | | | | | |
| | | X | 1 | 350000 | 51 | | 9 | 18 | 24 | |
| | | X | 2 | 370000 | 43 | 47 | 10 | 13 | 20 | |
| | | Y | 1 | 770000 | 73 | | 5 | 22 | 46 | |
| | | Y | 2 | 900000 | 81 | 77 | 7 | 17 | 57 | |
| 23 | Apr. 29, 2014 | BMA | 1 | 120000 | 48 | | 17 | 13 | 18 | |
| | | BMA | 2 | 130000 | 43 | 45.5 | 17 | 14 | 12 | |
| | | X | 1 | 950000 | 90 | | | | | Assigned 90 |
| | | X | 2 | 900000 | 90 | 90 | | | | Assigned 90 |
| | | Y | 1 | 550000 | 86 | | 12 | 26 | 48 | 86 but |
| | | | | | | | | | | inaccurate |
| | | Y | 2 | 590000 | 90 | 88 | | | | Assigned 90 |
| 24 | May 1, 2014 | BMA | 1 | 700000 | 24 | | 1 | 1 | 22 | |
| | | BMA | 2 | 510000 | 25 | 24.5 | 5 | 2 | 18 | |
| | | X | 1 | 1200000 | 90 | | | | | Assigned 90 |
| | | X | 2 | 1600000 | 90 | 90 | | | | Assigned 90 |
| | | Y | 1 | 2000000 | 90 | | | | | Assigned 90 |
| | | Y | 2 | 2000000 | 90 | 90 | | | | Assigned 90 |
| 25 | May 1, 2014 | BMA | 1 | too few to | 3 | | 1 | 1 | 1 | |
| | | | | count | | | | | | |
| | | BMA | 2 | too few to | 2 | 2.5 | 1 | 0 | 1 | |
| | | | | count | | | | | | |
| | | X | 1 | 38000 | 9 | | 3 | 2 | 4 | |
| | | X | 2 | 38000 | 7 | 8 | 2 | 4 | 1 | |
| | | Y | 1 | 100000 | 13 | | 6 | 4 | 3 | |
| | | Y | 2 | 100000 | 12 | 12.5 | 6 | 3 | 3 | |

TABLE 6
Jennifer Cassano/Dr. Fortier
Hu TGF-β Multiplex
Jun. 25, 2014

| | TGF-b1 (78) | | | TGF-b2 (33) | | | TGF-b3 (6) | | |
|---|---|---|---|---|---|---|---|---|---|
| | Exp | Obs | | Exp | Obs | | Exp | Obs | |
| Description | Conc | Conc | FI | Conc | Conc | FI | Conc | Conc | FI |
| Blank | | | 7.9 | | | 3.8 | | | 1.3 |
| Standard 1 | 26700 | 26700.51 | 5887 | 12900 | 12938.52 | 6930.5 | 45400 | 45522.33 | 3826 |
| Standard 2 | 8900 | 8897.34 | 2605.5 | 4300 | 4232.06 | 2837 | 15133.33 | 14995.09 | 1347 |
| Standard 3 | 2966.67 | 2998.25 | 886 | 1433.33 | 1494.78 | 1109 | 5044.44 | 5154.07 | 485 |
| Standard 4 | 988.89 | 953.16 | 288.5 | 477.78 | 453.59 | 342.5 | 1681.48 | 1600.41 | 157 |
| Standard 5 | 329.63 | 351.2 | 114.5 | 159.26 | 167.62 | 123.5 | 560.49 | 616.57 | 63 |
| Standard 6 | 109.88 | 104.69 | 44 | 53.09 | 51.73 | 39.5 | 186.83 | 175.33 | 20 |
| Standard 7 | 36.63 | 37.49 | 25 | 17.7 | 17.86 | 18 | 62.28 | 63.73 | 9 |

| | | |
|---|---|---|
| TGF-b1 (78) | TGF-b2 (33) | TGF-b3 (6) |

| | Obs | | Obs | | Obs | |
|---|---|---|---|---|---|---|
| Description | Conc | Fl | Conc | Fl | Conc | Fl |
| 8 BMA | OOR < | 13 | OOR < | 8 | OOR < | 1 |
| 9 | *171.92 | 19 | OOR < | 8 | OOR < | 1 |
| 10 | OOR < | 13 | OOR < | 7 | OOR < | 1 |
| 14 | OOR < | 13.5 | OOR < | 8 | OOR < | 1 |
| 15 | OOR < | 14 | OOR < | 7 | OOR < | 1 |
| 16 | 420.21 | 25.5 | OOR < | 8.5 | OOR < | 1 |
| 17 | OOR < | 12 | OOR < | 9 | OOR < | 1 |
| 18 | OOR < | 13 | OOR < | 7 | OOR < | 2 |
| 19 | OOR < | 10 | OOR < | 9 | OOR < | 1 |
| 20 | *56.21 | 16 | OOR < | 9 | OOR < | 1 |
| 21 | OOR < | 13 | OOR < | 7 | OOR < | 2 |
| 22 | *94.92 | 17 | OOR < | 7 | OOR < | 0 |
| 23 | *94.92 | 17 | OOR < | 9 | OOR < | 1 |
| 24 | OOR < | 13 | OOR < | 6 | OOR < | 1 |
| 25 | OOR < | 13 | OOR < | 8 | OOR < | 2 |
| 26 | OOR < | 11 | OOR < | 7 | OOR < | 1 |
| 27 | OOR < | 11 | OOR < | 7 | OOR < | 1 |
| 28 | OOR < | 13.5 | OOR < | 7 | OOR < | 0 |
| 29 | OOR < | 11 | OOR < | 7 | OOR < | 1 |
| 8 BMA C-X | *382.14 | 24.5 | OOR < | 9 | OOR < | 1 |
| 9 | *133.48 | 18 | OOR < | 7.5 | OOR < | 1 |
| 10 | *133.48 | 18 | OOR < | 7.5 | OOR < | 1.5 |
| 14 | *17.20 | 15 | OOR < | 8 | OOR < | 1 |
| 15 | *152.71 | 18.5 | OOR < | 6 | OOR < | 1 |
| 16 | *210.27 | 20 | OOR < | 7 | OOR < | 1 |
| 17 | OOR < | 13 | OOR < | 8 | OOR < | 1 |
| 18 | 439.23 | 26 | OOR < | 7 | OOR < | 1 |
| 19 | OOR < | 11 | OOR < | 6.5 | OOR < | 1 |
| 20 | 439.23 | 26 | OOR < | 6 | OOR < | 2 |
| 21 | OOR < | 12 | OOR < | 6.5 | OOR < | 1 |
| 22 | *248.56 | 21 | OOR < | 7 | OOR < | 1 |
| 23 | *133.48 | 18 | OOR < | 6 | OOR < | 1 |
| 24 | *171.92 | 19 | OOR < | 5 | OOR < | 1 |
| 25 | *286.78 | 22 | OOR < | 6 | OOR < | 1 |
| 26 | *17.20 | 15 | OOR < | 8 | OOR < | 2 |
| 27 | OOR < | 13 | OOR < | 6 | OOR < | 1 |
| 28 | *56.21 | 16 | OOR < | 7.5 | OOR < | 1 |
| 29 | OOR < | 14.5 | OOR < | 7 | OOR < | 1 |
| 8 BMA C-Y | *229.42 | 20.5 | OOR < | 5.5 | OOR < | 1 |
| 9 | *363.09 | 24 | OOR < | 7 | OOR < | 1 |
| 10 | *210.27 | 20 | OOR < | 6 | OOR < | 2 |
| 14 | *210.27 | 20 | OOR < | 6 | OOR < | 1 |
| 15 | OOR < | 12 | OOR < | 4 | OOR < | 1.5 |
| 16 | *363.09 | 24 | OOR < | 6.5 | OOR < | 2 |
| 17 | *56.21 | 16 | OOR < | 7 | OOR < | 1 |
| 18 | *286.78 | 22 | OOR < | 6 | OOR < | 1 |
| 19 | OOR < | 13 | OOR < | 6 | OOR < | 1 |
| 20 | *286.78 | 22 | OOR < | 8 | OOR < | 2 |
| 21 | OOR < | 10 | OOR < | 5 | OOR < | 1 |
| 22 | *210.27 | 20 | OOR < | 7 | OOR < | 1 |
| 23 | *56.21 | 16 | OOR < | 5.5 | OOR < | 1 |
| 24 | 666.94 | 32 | OOR < | 6 | OOR < | 2 |
| 25 | *248.56 | 21 | OOR < | 6 | OOR < | 1 |
| 26 | OOR < | 10 | OOR < | 5 | OOR < | 1 |
| 27 | OOR < | 13 | OOR < | 5 | OOR < | 1 |
| 28 | *17.20 | 15 | OOR < | 5 | OOR < | 1 |
| 29 | *94.92 | 17 | OOR < | 6 | OOR < | 1 |
| 16 PRP | *133.48 | 18 | OOR < | 7 | OOR < | 1 |
| 17 | OOR < | 11 | OOR < | 6 | OOR < | 2 |
| 18 | OOR < | 14 | OOR < | 5.5 | OOR < | 1 |
| 19 | OOR < | 9 | OOR < | 6 | OOR < | 1 |
| 20 | *56.21 | 16 | OOR < | 6 | OOR < | 1 |
| 21 | OOR < | 13 | OOR < | 6 | OOR < | 1 |
| 22 | *133.48 | 18 | OOR < | 6 | OOR < | 1 |
| 23 | OOR < | 14 | OOR < | 7 | OOR < | 2 |
| 24 | *17.20 | 15 | OOR < | 5.5 | OOR < | 1 |
| 25 | OOR < | 13 | OOR < | 7 | OOR < | 1 |
| 26 | OOR < | 11 | OOR < | 6 | OOR < | 1 |
| 27 | OOR < | 14 | OOR < | 5 | OOR < | 1 |
| 28 | *94.92 | 17 | OOR < | 5 | OOR < | 2 |
| 29 | *17.20 | 15 | OOR < | 4.5 | OOR < | 1 |
| 16 Whole Blood | OOR < | 13 | OOR < | 6 | OOR < | 1 |
| 17 | OOR < | 11 | OOR < | 6 | OOR < | 2 |
| 18 | OOR < | 14 | OOR < | 6 | OOR < | 2 |
| 19 | OOR < | 8.5 | OOR < | 5 | OOR < | 1 |
| 20 | *17.20 | 15 | OOR < | 6 | OOR < | 1 |
| 21 | OOR < | 12.5 | OOR < | 7 | OOR < | 1 |
| 22 | *210.27 | 20 | OOR < | 7 | OOR < | 2 |
| 23 | *17.20 | 15 | OOR < | 6 | OOR < | 2.5 |
| 24 | *94.92 | 17 | OOR < | 6 | OOR < | 2 |
| 25 | OOR < | 13.5 | OOR < | 6 | OOR < | 1 |
| 26 | *17.20 | 15 | OOR < | 6 | OOR < | 1 |
| 27 | *94.92 | 17 | OOR < | 6 | OOR < | 2 |
| 28 | *17.20 | 15 | OOR < | 5.5 | OOR < | 1 |
| 29 | OOR < | 12 | OOR < | 6.5 | OOR < | 1 |

### REFERENCES

1. Anz A W, Hackel J G, Nilssen E C, Andrews J R (2014) Application of biologics in the treatment of the rotator cuff, meniscus, cartilage, and osteoarthritis. J Am Acad Orthop Surg 22:68-79
2. Ataliotis P (2000) Platelet-derived growth factor A modulates limb chondrogenesis both in vivo and in vitro. Mech Dev 94:13-24
3. Baer P C, Geiger H (2012) Adipose-derived mesenchymal stromal/stem cells: tissue localization, characterization, and heterogeneity. Stem Cells Int 2012:812693
4. Baggiolini M, Clark-Lewis I (1992) Interleukin-8, a chemotactic and inflammatory cytokine. FEBS Lett 307:97-101
5. Baltzer A W, Moser C, Jansen S A, Krauspe R (2009) Autologous conditioned serum (Orthokine) is an effective treatment for knee osteoarthritis. Osteoarthritis Cartilage 17:152-160
6. Barry F, Murphy M (2013) Mesenchymal stem cells in joint disease and repair. Nat Rev Rheumatol 9:584-594
7. Barry F, Boynton R E, Liu B, Murphy J M (2001) Chondrogenic differentiation of mesenchymal stem cells from bone marrow: differentiation-dependent gene expression of matrix components. Exp Cell Res 268:189-200
8. Bazzoni F, Cassatella M A, Rossi F, Ceska M, Dewald B, Baggiolini M (1991) Phagocytosing neutrophils produce and release high amounts of the neutrophil-activating peptide 1/interleukin 8. J Exp Med 173:771-774
9. Blaney Davidson E N, van der Kraan P M, van den Berg W B (2007) TGF-beta and osteoarthritis. Osteoarthritis Cartilage 15:597-604
10. Boswell S G, Cole B J, Sundman E A, Karas V, Fortier L A (2012) Platelet-rich plasma: a milieu of bioactive factors. Arthroscopy 28:429-439
11. Centeno C J, Busse D, Kisiday J, Keohan C, Freeman M, Karli D (2008) Increased knee cartilage volume in degenerative joint disease using percutaneously implanted, autologous mesenchymal stem cells. Pain Physician 11:343-353
12. Chen J, Chen H, Li P, Diao H, Zhu S, Dong L, Wang R, Guo T, Zhao J, Zhang J (2011) Simultaneous regeneration of articular cartilage and subchondral bone in vivo using MSCs induced by a spatially controlled gene delivery system in bilayered integrated scaffolds. Biomaterials 32:4793-4805
13. Cole B J, Seroyer S T, Filardo G, Bajaj S, Fortier L A (2010) Platelet-rich plasma: where are we now and where are we going?. Sports Health 2:203-210
14. Cuthbert R, Boxall S A, Tan H B, Giannoudis P V, McGonagle D, Jones E (2012) Single-platform quality control assay to quantify multipotential stromal cells in bone marrow aspirates prior to bulk manufacture or direct therapeutic use. Cytotherapy 14:431-440
15. Dinarello C A, Thompson R C (1991) Blocking IL-1: interleukin 1 receptor antagonist in vivo and in vitro. Immunol Today 12:404-410
16. Dominici M, Le Blanc K, Mueller I, Slaper-Cortenbach I, Marini F, Krause D, Deans R, Keating A, Prockop D, Horwitz E (2006) Minimal criteria for defining multipotent mesenchymal stromal cells. The International Society for Cellular Therapy position statement. Cytotherapy 8:315-317
17. Enea D, Cecconi S, Calcagno S, Busilacchi A, Manzotti S, Kaps C, Gigante A (2013) Single-stage cartilage repair in the knee with microfracture covered with a resorbable polymer-based matrix and autologous bone marrow concentrate. Knee 20:562-569
18. Fan J, Ren L, Liang R, Gong Y, Cai D, Wang D A (2010) Chondrogenesis of synovium-derived mesenchymal stem cells in photopolymerizing hydrogel scaffolds. J Biomater Sci Polym Ed 21:1653-1667
19. Fernandes J C, Martel-Pelletier J, Pelletier J P (2002) The role of cytokines in osteoarthritis pathophysiology. Biorheology 39:237-246
20. Filardo G, Kon E, Roffi A, Di Matteo B, Merli M L, Marcacci M (2013) Platelet-rich plasma: why intra-articular? A systematic review of preclinical studies and clinical evidence on PRP for joint degeneration. Knee Surg Sports Traumatol Arthrosc
21. Filardo G, Madry H, Jelic M, Roffi A, Cucchiarini M, Kon E (2013) Mesenchymal stem cells for the treatment of cartilage lesions: from preclinical findings to clinical application in orthopaedics. Knee Surg Sports Traumatol Arthrosc 21:1717-1729
22. Fortier L A, Barker J U, Strauss E J, McCarrel T M, Cole B J (2011) The role of growth factors in cartilage repair. Clin Orthop Relat Res 469:2706-2715
23. Fortier L A, Potter H G, Rickey E J, Schnabel L V, Foo L F, Chong L R, Stokol T, Cheetham J, Nixon A J (2010) Concentrated bone marrow aspirate improves full-thickness cartilage repair compared with microfracture in the equine model. J Bone Joint Surg Am 92:1927-1937
24. Galotto M, Berisso G, Delfino L, Podesta M, Ottaggio L, Dallorso S, Dufour C, Ferrara G B, Abbondandolo A, Dini G, Bacigalupo A, Cancedda R, Quarto R (1999) Stromal damage as consequence of high-dose chemo/radiotherapy in bone marrow transplant recipients. Exp Hematol 27:1460-1466
25. Gharibi B, Hughes F J (2012) Effects of medium supplements on proliferation, differentiation potential, and in vitro expansion of mesenchymal stem cells. Stem Cells Transl Med 1:771-782
26. Gigante A, Cecconi S, Calcagno S, Busilacchi A, Enea D (2012) Arthroscopic knee cartilage repair with covered microfracture and bone marrow concentrate. Arthrosc Tech 1:e175-80
27. Gobbi A, Karnatzikos G, Scotti C, Mahajan V, Mazzucco L, Grigolo B (2011) One-step cartilage repair and bone marrow aspirate concentrated cells and collagen matrix in full-thickness knee cartilage lesions: results at 2-year follow-up. Cartilage 2:286-299
28. Gobbi A, Karnatzikos G, Sankineani S R (2014) One-step surgery with multipotent stem cells for the treatment of large full-thickness chondral defects of the knee. Am J Sports Med 42:648-657
29. Goldring M B (2000) Osteoarthritis and cartilage: the role of cytokines. Curr Rheumatol Rep 2:459-465
30. Granowitz E V, Clark B D, Mancilla J, Dinarello C A (1991) Interleukin-1 receptor antagonist competitively inhibits the binding of interleukin-1 to the type II interleukin-1 receptor. J Biol Chem 266:14147-14150
31. Grotendorst G R, Smale G, Pencev D (1989) Production of transforming growth factor beta by human peripheral blood monocytes and neutrophils. J Cell Physiol 140:396-402
32. Hattar K, Fink L, Fietzner K, Himmel B, Grimminger F, Seeger W, Sibelius U (2001) Cell density regulates neutrophil IL-8 synthesis: role of IL-1 receptor antagonist and soluble TNF receptors. J Immunol 166:6287-6293
33. Hegde V, Shonuga O, Ellis S, Fragomen A, Kennedy J, Kudryashov V, Lane J M (2014) A prospective comparison of 3 approved systems for autologous bone marrow concentration demonstrated nonequivalency in progenitor cell number and concentration. J Orthop Trauma 28:591-598
34. Johnson R G (2014) Bone marrow concentrate with allograft equivalent to autograft in lumbar fusions. Spine (Phila Pa. 1976) 39:695-700
35. Johnstone B, Hering T M, Caplan A I, Goldberg V M, Yoo J U (1998) In vitro chondrogenesis of bone marrow-derived mesenchymal progenitor cells. Exp Cell Res 238:265-272
36. Koh Y G, Choi Y J, Kwon O R, Kim Y S (2014) Second-Look Arthroscopic Evaluation of Cartilage Lesions After Mesenchymal Stem Cell Implantation in Osteoarthritic Knees. Am J Sports Med 42:1628-1637
37. Kurth T, Hedbom E, Shintani N, Sugimoto M, Chen F H, Haspl M, Martinovic S, Hunziker E B (2007) Chondrogenic potential of human synovial mesenchymal stem cells in alginate. Osteoarthritis Cartilage 15:1178-1189
38. Lv F J, Tuan R S, Cheung K M, Leung V Y (2014) Concise review: the surface markers and identity of human mesenchymal stem cells. Stem Cells 32:1408-1419
39. Mackay A M, Beck S C, Murphy J M, Barry F P, Chichester C O, Pittenger M F (1998) Chondrogenic differentiation of cultured human mesenchymal stem cells from marrow. Tissue Eng 4:415-428
40. McCarrel T, Fortier L (2009) Temporal growth factor release from platelet-rich plasma, trehalose lyophilized platelets, and bone marrow aspirate and their effect on tendon and ligament gene expression. J Orthop Res 27:1033-1042
41. Meijer H, Reinecke J, Becker C, Tholen G, Wehling P (2003) The production of anti-inflammatory cytokines in whole blood by physico-chemical induction. Inflamm Res 52:404-407
42. Montaseri A, Busch F, Mobasheri A, Buhrmann C, Aldinger C, Rad J S, Shakibaei M (2011) IGF-1 and PDGF-bb suppress IL-1beta-induced cartilage degradation through down-regulation of NF-kappaB signaling: involvement of Src/PI-3K/AKT pathway. PLoS One 6:e28663
43. Mueller M B, Tuan R S (2008) Functional characterization of hypertrophy in chondrogenesis of human mesenchymal stem cells. Arthritis Rheum 58:1377-1388
44. Mwale F, Stachura D, Roughley P, Antoniou J (2006) Limitations of using aggrecan and type X collagen as markers of chondrogenesis in mesenchymal stem cell differentiation. J Orthop Res 24:1791-1798
45. Poloni A, Maurizi G, Rosini V, Mondini E, Mancini S, Discepoli G, Biasio S, Battaglini G, Felicetti S, Berardinelli E, Serrani F, Leoni P (2009) Selection of CD271(+) cells and human AB serum allows a large expansion of mesenchymal stromal cells from human bone marrow. Cytotherapy 11:153-162
46. Rutgers M, Saris D B, Dhert W J, Creemers L B (2010) Cytokine profile of autologous conditioned serum for treatment of osteoarthritis, in vitro effects on cartilage metabolism and intra-articular levels after injection. Arthritis Res Ther 12:R114
47. Schnabel L V, Mohammed H O, Miller B J, McDermott W G, Jacobson M S, Santangelo K S, Fortier L A (2007) Platelet rich plasma (PRP) enhances anabolic gene expression patterns in flexor digitorum superficialis tendons. J Orthop Res 25:230-240
48. Skowronski J, Rutka M (2013) Osteochondral lesions of the knee reconstructed with mesenchymal stem cells—results. Ortop Traumatol Rehabil 15:195-204
49. Tchetina E V, Antoniou J, Tanzer M, Zukor D J, Poole A R (2006) Transforming growth factor-beta2 suppresses collagen cleavage in cultured human osteoarthritic cartilage, reduces expression of genes associated with chondrocyte hypertrophy and degradation, and increases prostaglandin E(2) production. Am J Pathol 168:131-140
50. Tsutsumi S, Shimazu A, Miyazaki K, Pan H, Koike C, Yoshida E, Takagishi K, Kato Y (2001) Retention of multilineage differentiation potential of mesenchymal cells during proliferation in response to FGF. Biochem Biophys Res Commun 288:413-419
51. Tuli R, Tuli S, Nandi S, Huang X, Manner P A, Hozack W J, Danielson K G, Hall D J, Tuan R S (2003) Transforming growth factor-beta-mediated chondrogenesis of human mesenchymal progenitor cells involves N-cadherin and mitogen-activated protein kinase and Wnt signaling cross-talk. J Biol Chem 278:41227-41236
52. Van Lent P L, Van De Loo F A, Holthuysen A E, Van Den Bersselaar L A, Vermeer H, Van Den Berg W B (1995) Major role for interleukin 1 but not for tumor necrosis factor in early cartilage damage in immune complex arthritis in mice. J Rheumatol 22:2250-2258
53. Veronesi F, Giavaresi G, Tschon M, Borsari V, Nicoli Aldini N, Fini M (2013) Clinical use of bone marrow, bone marrow concentrate, and expanded bone marrow mesenchymal stem cells in cartilage disease. Stem Cells Dev 22:181-192
54. Wehling P, Moser C, Frisbie D, Mcllwraith C W, Kawcak C E, Krauspe R, Reinecke J A (2007) Autologous conditioned serum in the treatment of orthopedic diseases: the orthokine therapy. BioDrugs 21:323-332
55. Yang K G, Raijmakers N J, van Arkel E R, Caron J J, Rijk P C, Willems W J, Zijl J A, Verbout A J, Dhert W J, Saris D B (2008) Autologous interleukin-1 receptor antagonist improves function and symptoms in osteoarthritis when compared to placebo in a prospective randomized controlled trial. Osteoarthritis Cartilage 16:498-505
56. Yoo J U, Barthel T S, Nishimura K, Solchaga L, Caplan A I, Goldberg V M, Johnstone B (1998) The chondrogenic potential of human bone-marrow-derived mesenchymal progenitor cells. J Bone Joint Surg Am 80:1745-1757

1. A method for preparing a bone marrow concentrate, comprising
(1) collecting a bone marrow sample in a subject; and
(2) concentrating the bone marrow sample, thereby preparing the bone marrow concentrate, wherein the bone marrow concentrate comprises an autologous interleukin receptor antagonist protein (TRAP) concentrate comprising at least about 10,000 pg/mL of TRAP.

2. The method of claim 1, wherein the concentrating step is performed via centrifugation.

3. The method of claim 2, wherein the centrifugation is performed with a sensor.

4. The method of claim 3, wherein the sensor is a light sensor or a laser sensor that detects fluid density change.

5. The method of claim 1, wherein the RAP has a concentration of at least about 12,000 pg/mL, about 15,000 pg/mL or more.

6. The method of claim 1, wherein the bone marrow concentrate further comprises a cytokine.

7. The method of claim 6, wherein the cytokine is PDGF, TGF-β1, TGF-β2, TGF-β3, IL-1β, IL-8, or any combination thereof.

8. The method of claim 1, wherein the bone marrow sample is collected in the presence of at least one anticoagulant agent.

9. The method of claim 8, wherein the anticoagulant agent comprises acid citrate dextrose solution (ACD) or Heparin.

10. A method for facilitating soft tissue healing in a subject in need thereof, comprising administering to the subject a therapeutically effective amount of a composition comprising the bone marrow concentrate prepared according to claim 1.

11. The method of claim 10, wherein the composition is administered locally.

12. The method of claim 11, wherein the composition is administered via local injection.

13. The method of claim 10, wherein the soft tissue is cartilage.

14. The method of claim 10, further comprising locally administering to the subject a therapeutically effective amount of a second composition comprising a platelet rich plasma (PRP).

15. A method for treating osteoarthritis in a subject in need thereof, comprising administering to the subject a therapeutically effective amount of a composition comprising the bone marrow concentrate prepared according to claim 1.

16. The method of claim 15, wherein the composition is administered locally.

17. A method for facilitating skin regeneration a subject in need thereof, comprising administering to the subject a therapeutically effective amount of a composition comprising the bone marrow concentrate prepared according to claim 1.

18. The method of claim 17, wherein the composition is administered locally.

19. A method for treating inflammation in a subject in need thereof, comprising administering to the subject a therapeutically effective amount of a composition comprising the bone marrow concentrate prepared according to claim 1.

20. The method of claim 19, wherein the composition is administered locally.

21. A composition comprising a bone marrow concentrate, wherein the bone marrow concentrate is prepared according to claim 1.
